(19) United States
(12) Patent Application Publication
(10) Pub. No.: US 2023/0355095 A1
(43) Pub. Date: Nov. 9, 2023

(54) TREATMENT CHAIR

(71) Applicant: KURE, LLC, Newark, DE (US)

(72) Inventors: Yale BADIK, MADISON, CT (US); Deanna FELKER, West Hills, CA (US)

(21) Appl. No.: 17/739,463

(22) Filed: May 9, 2022

(51) Int. Cl. A61B 5/00; G16H 40/63; G16H 80/00; A61G 7/002; A61G 7/075

(52) U.S. Cl. CPC A61B 5/002; A61B 5/6891; G16H 40/63; G16H 80/00; A61G 7/002; A61G 7/075

(57) ABSTRACT

Embodiments relate to an article of furniture comprising a drug delivery system; a support surface to support at least a portion of a living body; an enclosure to surround at least the portion of the living body; a bio-monitoring system to monitor a physiological state of the living body, a method of providing a therapy to a person using the device, and a method for estimating an effect of the therapy through the device.

### RELATED APPLICATIONS

The present invention is related to U.S. patent applications bearing attorney docket number KURE-002-00US (application Ser. No. 17/739,588; filed on May 9, 2022) entitled SMART EYE MASK; KURE-003-00US (application Ser. No. 17/739,673; filed on May 9, 2022) entitled INFUSION AND MONITORING SYSTEM; KURE-004-00US (application Ser. No. 17/739,756; filed on May 9, 2022) entitled SMART STORAGE SYSTEM and KURE-005-00US (application Ser. No. 17/739,835; filed on May 9, 2022) entitled SMART DISPENSER which are being concurrently filed. All U.S. patent applications referred above are incorporated, for the purposes of written description, herein by reference in their entirety.

### FIELD OF THE INVENTION

This invention relates generally to chairs. More particularly, the present invention relates to a multi-stimuli chair for therapy.

### BACKGROUND

“A system and method utilizing a synchronized color, sound, and aroma therapy is described. The present system and methods synchronize frequencies of color, light, aroma, and sound to provide a sensory experience. This guided visual, auditory, and aromatic stimulation is intended to alleviate stress and/or anxiety by relaxing and stimulating the brain, thereby correcting any neurophysical imbalances. Reduction in the levels of stress and/or anxiety is achieved through use of the auditory, visual, and aromatic sequences which are individually specific and systematic.” [Source: System and method for reducing stress levels using color, sound, and aroma therapy; Oyvind Berg and Elisabeth Rosse; issued as U.S. Pat. No. 9,839,762B2 on 12 Dec. 2017]

“Multisensory activity is currently performed only in specific centers provided with suitably equipped rooms known as “Snoezelen Rooms.” Such rooms are designed by seeking an optimization of one or more environmental elements (screens for external light, colors of walls, ceilings and floors, textures of surfaces); visual elements (disk image projectors, light effects); tactile elements (vibrating musical armchairs); musical elements (specifically selected lounge music); and olfactory elements (aromas in the environment) . . . . The “Snoezelen Room”, due to the complexity of the parameters, is in fact particularly expensive both as far as design is concerned and during use. This problem causes this type of therapy to be particularly expensive and therefore not accessible to everyone. Another problem linked to the above cited high costs is that, despite the countless benefits of multisensory stimulation, the diffusion of these therapies is extremely limited. A further problem is constituted by adjustment, which due to the large number of parameters is complex even for a skilled technician.” [Source: Device for multisensory stimulation; Leila Benedicte Habiche; published as US20130261378A1 on 3 Oct. 2013]

“Hagivara et al., U.S. Pat. No. 5,024,650, entitled Stress Dissolving Refreshment System, is representative of systems utilized in the prior art to create a relaxation state or environment for the user. The system requires the user to be in a reclining chair which is surrounded by a variety of stimuli. The '650 system is bulky, complex, and cannot be fittable or movable through a standard doorway. Although the '650 system provides several stimuli, it does not provide magnets (or a magnetic field) or variably colored lighting.” [Source: Multi-stimuli chair; Gary L. Moses; published as U.S. Pat. No. 5,387,178A on 7 Feb. 1995]

“The portable relaxation pod includes a foldable exterior housing with walls, front flaps configured to close at least partially and an interior ceiling. Interior panels have acoustic foam and corrugated plastic layers. The interior panels abut the ceiling and walls. The portable seating system of a foldable chair equipped with a massage cushion is placed inside the exterior housing. A relaxation system includes an eye mask, headphones connected to an audio player and a portable foot massager, which is placed outside of the exterior housing in front of the seating system.” [Source: Portable relaxation pod; Annemarie Ruggiero; issued as U.S. Ser. No. 10/837,194B2 on 17 Nov. 2020]

“A system in which a wearable device detects an electroencephalographic (EEG) response from a user during sleep-related activity, e.g., trying to fall asleep, being asleep or waking up, and outputs an audio signal that is tailored, based on the EEG response, to enhance the user's sleep experience. In particular, an audio, thermal and/or olfactory signal may be used to facilitate any one or more of (i) a smooth path into REM sleep, (ii) a reduced potential for sleep being disturbed, and (iii) exit from sleep at an optimum time.” [Source: Sleep enhancement system and wearable device for use therewith; Harrison and Morgan; published as US20200261689A1 on 20 Aug. 2020]

“[T]he sensor was capable to detect various movements of eyelid and eyeballs from the most-comfortably-wearable temple area. This noninvasive, nontoxic, and easily-wearable eye movement sensor can detect eye blink frequency, blink duration, and percent of eye closure to function as an objective indicator of eye strain, fatigue, and drowsiness. The sensor can also measure lateral movements of eyeballs, which are distinguished from the eye blink . . . ” [Source: Kim et. al., Highly-Sensitive Skin-Attachable Eye-Movement Sensor Using Flexible Nonhazardous Piezoelectric Thin Film. Adv. Funct. Mater. 2021, 31, 2008242.]

Considering the knowledge of persons skilled in the art, there is a long-felt need to address the shortcomings in the prior art and provide a system that is capable of providing a comfortable uninterruptible environment for therapy. It would be advantageous to have an apparatus that considers at least some of the issues discussed above, as well as possibly other issues.

### SUMMARY

The present disclosure describes one or more aspects of providing a comfortable article of furniture that provides an uninterruptible environment for therapy and is also capable of aiding in the therapy.

In an aspect a device is described herein. The device comprises a drug delivery system; a support surface to support at least a portion of a living body; an enclosure to surround at least the portion of the living body; a bio-monitoring system to monitor a physiological state of the living body.

In an embodiment, the device comprises an article of furniture.

In another embodiment, the support surface further comprises a blanket.

In yet another embodiment, the blanket is at least one of a weighted blanket, a grounding mat, a magnetic therapy pad and a temperature adjustable blanket.

In yet another embodiment, the device further comprises an armrest, wherein the armrest is foldable.

In yet another embodiment, the armrest is provided with a heating pad.

In yet another embodiment, the enclosure comprises an adjustable dome shaped housing that allows freedom of movement.

In yet another embodiment, the adjustable dome shaped housing further comprises a bridge, wherein the bridge is foldable.

In yet another embodiment, the bridge comprises a visual display.

In yet another embodiment, the bio-monitoring system comprises a vital monitoring system.

In yet another embodiment, the vital monitoring system comprises at least one of an EEG system, an ultrasound transducer, a heart rate monitor, a breathing monitor, and a pulse rate monitor.

In yet another embodiment, the bio-monitoring system further comprises at least one of a body motion monitoring system, a brain wave monitoring system, a facial expression recognizing system and an eye monitoring system.

In yet another embodiment, the bio-monitoring system comprises a biofeedback control system and wherein an output from the biofeedback control system controls a delivery of a drug by the drug delivery system.

In yet another embodiment, the device further comprises an attachable device.

In yet another embodiment, the attachable device comprises at least one of a sound emitting device, a light emitting device, an aroma infusion device, an eye mask, and a computing system.

In yet another embodiment, the attachable device is connected to the device through one of a wireless and a wired connection.

In yet another embodiment, the sound emitting device is one of an ear pod, a 360-degree sound speaker and a headphone.

In yet another embodiment, the sound emitting device is operable to provide one of a programmed meditation audio, a brain stimulating wave frequency, a music, and a nature sound.

In yet another embodiment, the light emitting device provides a predetermined light frequency for a predetermined duration.

In yet another embodiment, the computing system can remotely take and execute a control instruction.

In yet another embodiment, the computing system is at least one of a smartphone, a tablet, a laptop, a desktop, and a built-in computer.

In yet another embodiment, the device further comprises a control unit, wherein the control unit comprises an input module, a processor, a communication module, a database, a universal serial bus, a controller, a display, and a power module and wherein the control unit can remotely take and execute a control instruction.

In yet another embodiment, the power module further comprises one or more of a rechargeable battery, a non-rechargeable battery, a solar cell, a chemical reaction power generator, a power input port that connects to an external power line, or any other device configured to provide power to components of the device.

In yet another embodiment, the power module is arranged and disposed to provide wireless charging through an induction charger.

In yet another embodiment, the device can communicate data to a server via the communication module.

In yet another embodiment, the control unit further comprises a cyber security module.

In yet another embodiment, the cyber security module further comprises an information security management module providing isolation between the device and the server.

In yet another embodiment, the information security management module is operable to: receive data from at least one of the input module, the bio-monitoring system, the attachable device, the drug delivery system, and the database; exchange a security key at a start of the communication between the communication module and the server; receive the security key from the server; authenticate an identity of the server by verifying the security key; analyze the security key for a potential cyber security threat; negotiate an encryption key between the communication module and the server; encrypt the data; and transmit the encrypted data to the server when no cyber security threat is detected.

In yet another embodiment, the information security management module is operable to: exchange a security key at a start of the communication between the communication module and the server; receive the security key from the server; authenticate an identity of the server by verifying the security key; analyze the security key for a potential cyber security threat; negotiate an encryption key between the system and the server; receive encrypted data; decrypt the encrypted data; perform an integrity check of the decrypted data; and transmit the decrypted data to at least one of the input module, the bio-monitoring system, the drug delivery system, and the database through the communication module when no cyber security threat is detected.

In yet another embodiment, the information security management module is configured to raise an alarm when the cyber security threat is detected.

In yet another embodiment, the information security management module is configured to discard the encrypted data received if the integrity check of the encrypted data fails.

In yet another embodiment, the information security management module is configured to check the integrity of the encrypted data by checking accuracy, consistency, and any possible data loss during the communication through the communication module.

In yet another embodiment, the information security management module is configured to perform asynchronous authentication and validation of the communication between the communication module and the server.

In yet another embodiment, a perimeter network provides an extra layer of protection.

In yet another embodiment, the perimeter network protects the system from a cyber security threat by using a plurality of firewalls.

In yet another embodiment, the device can be adjusted to an upright position, a reclined position, and a horizontal position.

In yet another embodiment, the device further comprises one or more storage spaces.

In yet another embodiment, the device further comprises wheels.

In yet another embodiment, the wheels are provided with wheel stoppers.

In yet another embodiment, the device further comprises a handlebar.

In yet another embodiment, the device has an autonomous navigation module to navigate autonomously on a predefined track.

In yet another embodiment, the living body comprises a human body or an animal body.

In an aspect a method is described herein. The method comprises steps of: providing a therapy to a living body using a device; monitoring a physiological state of the living body using the device; and estimating an effect of the therapy using a bio-monitoring system; and modifying the therapy based on the change in the physiological state; and wherein the device comprises an article of furniture comprising a support surface to support at least a portion of a living body; an enclosure to surround at least the portion of the living body; and the bio-monitoring system that monitors a change in the physiological state of the living body.

In an embodiment, the therapy is at least one of a psychedelic drug therapy, a chemotherapy, a nutrient infusion therapy, a sound therapy, an aromatherapy, a magnetic therapy, a massage therapy, a pressure therapy, a light therapy, and a visual based stress relief therapy.

In another embodiment, the sound therapy is provided using a sound emitting device.

In yet another embodiment, the sound emitting device can be remotely controlled to provide an audio stimulus.

In yet another embodiment, the audio stimulus is one of a programmed meditation audio, a brain stimulating frequency, a music, and a nature sound.

In yet another embodiment, the aromatherapy is provided using one of a built-in aroma infusion device and an aroma pouch.

In yet another embodiment, the massage therapy and the pressure therapy are provided using a blanket.

In yet another embodiment, the light therapy is provided using a light emitting device.

In yet another embodiment, the light emitting device is operable to output a predetermined light frequency and duration.

In yet another embodiment, the psychedelic drug therapy is done using a psychedelic drug, wherein the psychedelic drug is one of a ketamine, esketamine, methylenedioxy-methylamphetamine, psilocybin, cannabis, an antidepressant, an anti-anxiety drug, an antipsychotic, and a psychoactive drug.

In yet another embodiment, the visual based stress relief therapy is provided using a visual display.

In yet another embodiment, the bio-monitoring system comprises a biofeedback control system and a vital monitoring system.

In yet another embodiment, the biofeedback control system modifies and controls delivery of a drug to the living body.

In yet another embodiment, the method further comprises steps of storing data from a control unit of the device to a database; securing data access using a cyber security module; accessing the data from the database from a remote location via the cyber security module through authentication; and sending an instruction to the living body or a care provider via a communication module.

In an aspect, a method is described herein. The method comprises steps of providing a therapy to a living body via an article of furniture; and estimating an effect of the therapy on the living body via the article of furniture; wherein the article of furniture comprises a bio-monitoring system that monitors a physiological state of the living body.

In an embodiment, the therapy is provided using a therapeutic stimulus via the article of furniture.

In another embodiment, the therapeutic stimulus is at least one of a psychedelic drug, a chemotherapy, a nutrient infusion, an audio stimulus, an aroma stimulus, a touch stimulus, a visual stimulus, and a light stimulus.

In an aspect a device is described herein. The device comprises a support surface to support at least a portion of a living body; means for providing a therapeutic stimulus; an enclosure to surround at least the portion of the living body; a bio-monitoring system; and wherein the bio-monitoring system monitors a physiological state of the living body.

In yet another embodiment, the psychedelic drug, the chemotherapy, and the nutrient infusion are provided using a drug delivery system.

In yet another embodiment, the drug delivery system is monitorable and controllable from a remote location.

In yet another embodiment, the audio stimulus is provided using a sound emitting device.

In yet another embodiment, the audio stimulus is one of a programmed meditation audio, a brain stimulating frequency, a music, and a nature sound.

In yet another embodiment, the aroma stimulus is provided using one of a built-in aroma infusion device and an aroma pouch.

In yet another embodiment, the magnetic field is provided via one of a far infrared heat, pulsed electromagnetic field, red light therapy, negative ions, and natural crystals.

In yet another embodiment, the touch stimulus is provided using the support surface.

In yet another embodiment, the light stimulus is provided using a light emitting device.

In yet another embodiment, the enclosure further comprises a bridge, wherein the bridge is foldable.

In yet another embodiment, the device further comprises an eye mask attachment.

In an aspect a device is described herein. The device comprises a support surface to support at least a portion of a living body; an enclosure to surround at least the portion of the living body; the enclosure comprising a visual display; and a bio-monitoring system; and wherein the bio-monitoring system monitors a physiological state of the living body.

In another embodiment, the blanket is at least one of a weighted blanket, a grounding mat, and a temperature adjustable blanket.

In yet another embodiment, the visual display provides a pictorial display containing imagery designed to create relaxation and reduce stress.

In yet another embodiment, the device is configured to provide a therapeutic stimulus.

### BRIEF DESCRIPTION OF THE FIGURES

In the present disclosure, reference is made to the accompanying drawings, which form a part hereof. In the drawings, similar symbols typically identify similar components, unless context dictates otherwise. Various embodiments described in the detailed description, and drawings, are illustrative and not meant to be limiting. Other embodiments may be used, and other changes may be made, without departing from the spirit or scope of the subject matter presented herein. It will be understood that the aspects of the present disclosure, as generally described herein, and illustrated in the Figures, can be arranged, substituted, combined, separated, and designed in a wide variety of different configurations, all of which are contemplated herein. The embodiments are illustrated by way of example and not limitation in the figures of the accompanying drawings, in which like references indicate similar elements and in which:

FIG. 1 depicts the device comprising a chair, in one or more embodiments.

FIG. 2 depicts a support surface of the chair comprising a blanket, in one or more embodiments.

FIG. 3 depicts a storage space of the chair, in one or more embodiments.

FIG. 4A depicts a closed enclosure of the chair, in one or more embodiments.

FIG. 4B depicts an open enclosure of the chair, in one or more embodiments.

FIG. 4C depicts a partially open enclosure of the chair, in one or more embodiments.

FIG. 5A depicts a bridge of the chair comprising a light emitting device, in one or more embodiments.

FIG. 5B depicts a bridge of the chair comprising a visual display, in one or more embodiments.

FIG. 6A depicts a chair adjusted in an upright position, in one or more embodiments.

FIG. 6B depicts a chair adjusted in a reclined position, in one or more embodiments.

FIG. 7A depicts a sound emitting device as an attachable device of the chair, in one or more embodiments.

FIG. 7B depicts a light emitting device as an attachable device of the chair, in one or more embodiments.

FIG. 7C depicts an aroma emitting device as an attachable device of the chair, in one or more embodiments.

FIG. 8 depicts a control unit of the object, in one or more embodiments.

FIG. 9A provides a flow chart of providing a sound therapy and estimating an effect using the device, in one or more embodiments.

FIG. 9B provides a flow chart of providing an aromatherapy and estimating an effect using the device, in one or more embodiments.

FIG. 9C provides a flow chart of infusing a psychedelic drug to a person and estimating an effect using the device, in one or more embodiments.

FIG. 9D provides a flow chart of providing an aromatherapy in combination with a sound therapy using the device, in one or more embodiments.

FIG. 9E provides a flow chart of providing a psychedelic drug therapy in combination with an aromatherapy using the device, in one or more embodiments.

FIG. 9F provides a flow chart of providing a psychedelic drug therapy in combination with a sound therapy using the device, in one or more embodiments.

FIG. 9G provides a flow chart for providing a psychedelic drug in combination with a sound therapy and an aromatherapy using the device, in one or more embodiments.

FIG. 10A shows a block diagram of the cyber security module in view of the system and server.

FIG. 10B shows an embodiment of the cyber security module.

FIG. 10C shows another embodiment of the cyber security module.

FIG. 11 is an example system where a system hardening strategy may be implemented according to an embodiment of the invention.

FIG. 12 shows an architecture of a network using a transparent proxy in an Enterprise network as per an aspect of an embodiment of the present invention for active malware detection.

FIG. 13A illustrates a system for providing a virtual browsing environment according to an aspect of an embodiment of the invention.

FIG. 13B illustrates a computer included in the system of FIG. 13A, according to an embodiment of the invention.

FIG. 14 is a block diagram of a virtual machine architecture of an aspect of an embodiment of the present invention to prevent malicious software attack.

FIG. 15 is a block diagram for securing sensitive data associations for related data values of an aspect of an embodiment of the present invention.

FIG. 16 is a system block diagram showing an example client interacting with k+1 servers that allows a user to define their encryption and relieves a user from the task of managing keys used for data security, as per an aspect of an embodiment of the present invention.

FIG. 17 is a flow diagram describing a method for determining at least part of a network attack according to an embodiment of the present invention.

FIG. 18 depicts a flow diagram for a computer readable storage medium demonstrating instructions that cause the processor to perform a method for identifying locations to deploy intrusion detection system (IDS) Sensors within a network infrastructure, as per an aspect of an embodiment of the present invention.

FIG. 19 provides a flow chart of estimating an effect of the therapy on the living body via the article of furniture, in one or more embodiments.

### DETAILED DESCRIPTION

### Definitions and General Techniques

For simplicity and clarity of illustration, the drawing figures illustrate the general manner of construction. Descriptions and details of well-known features and techniques may be omitted to avoid unnecessarily obscuring the present disclosure. The dimensions of some of the elements in the figures may be exaggerated relative to other elements to help improve understanding of embodiments of the present disclosure. The same reference numeral in different figures denotes the same elements.

Although the following detailed description contains many specifics for the purpose of illustration, a person of ordinary skill in the art will appreciate that many variations and alterations to the following details can be made and are considered to be included herein.

Accordingly, the following embodiments are set forth without any loss of generality to, and without imposing limitations upon, any claims set forth. It is also to be understood that the terminology used herein is for the purpose of describing particular embodiments only and is not intended to be limiting. Unless defined otherwise, all technical and scientific terms used herein have the same meaning as commonly understood by one with ordinary skill in the art to which this disclosure belongs.

The articles “a” and “an” are used herein to refer to one or to more than one (i.e., to at least one) of the grammatical object of the article. By way of example, “an element” means one element or more than one element.

The terms “first”, “second”, “third”, and the like in the description and in the claims, if any, are used for distinguishing between similar elements and not necessarily for describing a particular sequence or chronological order. It is to be understood that the terms so used are interchangeable under appropriate circumstances such that the embodiments described herein are, for example, capable of operation in sequences other than those illustrated or otherwise described herein. Furthermore, the terms “include”, “have”, and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left”, “right”, “front”, “back”, “top”, “bottom”, “over”, “under” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element act, or instruction used herein should be construed as critical or essential unless explicitly described as such. Furthermore, as used herein, the term “set” is intended to include items (e.g., related items, unrelated items, a combination of related items and unrelated items, etc.), and may be used interchangeably with “one or more”. Where only one item is intended, the term “one” or similar language is used. Also, as used herein, the terms “has”, “have”, “having”, or the like are intended to be open-ended terms. Further, the phrase “based on” is intended to mean “based, at least in part, on” unless explicitly stated otherwise.

It should be understood that the terms “system,” “device,” “unit,” and/or “module” are used in this disclosure to refer to a different component, component, portion, or component of the different levels of the order. However, if other expressions may achieve the same purpose, these terms may be replaced by other expressions.

The terms “couple”, “coupled”, “couples”, “coupling”, and the like should be broadly understood and refer to as connecting two or more elements mechanically, electrically, and/or otherwise. Two or more electrical elements may be electrically coupled together, but not be mechanically or otherwise coupled together. Coupling may be for any length of time, e.g., permanent, or semi-permanent or only for an instant. “Electrical coupling” includes electrical coupling of all types. The absence of the word “removably”, “removable”, and the like near the word “coupled”, and the like does not mean that the coupling, etc. in question is or is not removable.

As defined herein, two or more elements or modules are “integral” or “integrated” if they operate functionally together. As defined herein, two or more elements are “non-integral” if each element can operate functionally independently.

As defined herein, “real-time” can, in some embodiments, be defined with respect to operations conducted as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All variations which come within the meaning and range of equivalency of the claims are to be embraced within their scope.

As defined herein, “approximately” can mean within a specified or unspecified range of the specified or unspecified stated value. In some embodiments, “approximately” can mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Even though particular combinations of features are disclosed in the specification, these combinations are not intended to limit the disclosure of possible implementations.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

An initial overview of technology embodiments is provided below, and specific technology embodiments are then described in further detail. This initial summary is intended to aid readers in understanding the technology more quickly but is not intended to identify key or essential technological features, nor is it intended to limit the scope of the claimed subject matter.

The embodiments herein and the various features and advantageous details thereof are explained more fully with reference to the non-limiting embodiments that are illustrated in the accompanying drawings and detailed in the following description.

In order to fully understand the scope of the invention, the following terms used herein are hereby defined.

As referred herein, “article of furniture” refers to those movable articles that are used to support people or things to sit or lie down.

As referred herein, “therapy” refers to a process of treating a physical disorder, a psychological disorder, or a mental distress through use of one or more stimuli. In an embodiment, the one or more stimuli comprise a drug, an audio stimulus, an aroma stimulus, a touch stimulus, a visual stimulus, and a light stimulus.

As referred herein, “drug” is a substance (other than food) that is used to prevent, diagnose, treat, enhance, inhibit, or relieve symptoms of a disease or abnormal condition. Some drugs may be used for psychedelic reasons which may not be necessary but are wanted. Some drugs may inhibit memory (for example, ketamine) for a period or enhance unconscious activity, to reduce hallucinations.

As referred herein, “aromatherapy” is a form of alternative or complimentary therapy in which essential oils or other scents are inhaled to achieve therapeutic benefit. It also comprises a range of traditional, alternative, or complementary therapies that use essential oils and other aromatic plant compounds.

As referred herein, “aroma infusion device” is a device that can diffuse aroma into the environment. The aroma infusion device can be selected from an electric aroma lamp diffuser, an oil lamp, air humidifier, essential oil diffuser aroma lamp or the like.

As referred herein, “attachable device” is a device capable of connecting with at least one attached device. The attachable device and the attached device are electrically interconnected. The connection can be a wired connection or wireless connection. The attachable device can support signal capturing, signal processing, signal transmission, signal display, signal storage and/or power provision. The signals can be, for example, analog or digital signals. The attachable device can, for example, be used to provide audio output and/or audio pick-up.

As referred herein, “blanket” is a swath of soft cloth large enough either to cover or to enfold most body parts of the living body.

As referred herein, “wearable” is used for a category of electronic devices that can be worn as accessories, embedded in clothing, implanted in the user's body, or even tattooed on the skin. An example of wearable technology is a disposable skin patch with sensors that transmit patient data wirelessly to a nearby control device. Another example could be fitness trackers, often in the form of wristbands or straps, that monitor things like physical activity and vital signs. Trackers may connect wirelessly to an app for data storage, processing, and reporting.

As referred herein, “bio-monitoring system” is the devices and programs connected to electrical sensors that help in receiving information about physiological and mental state of a wearer's body. The information comprises one or more of Electromyograph (EMG), Thermal biofeedback, neurofeedback/electroencephalograph (EEG), Electrodermograph (EDG), heat flux, pneumograph, capnometry data, hemoencephalography, and photoplethysmography (PPG).

In an embodiment, the bio-monitoring system can be a portable and wearable solution.

As referred herein, “feedback” is output of a system that is routed back as inputs as part of a chain of cause-and-effect that forms a circuit or loop. The system can then be said to feed back into itself. A feedback mechanism is a loop system in which the system responds to perturbation either in the same direction (positive feedback) or in the opposite direction (negative feedback). In a system, a feedback mechanism involves a process, a signal, or a mechanism that tends to initiate (or accelerate) or to inhibit (or slow down) a process. As an example, when a drug infusion slows down the heart rate or drops a body temperature, the drug delivery rate is reduced or stopped to improve the patient's physiological state.

As referred herein, “biofeedback control system”, is a system that controls or improves a treatment plan by making subtle changes, based on the information provided by the bio-monitoring system, to improve a health condition, a physical performance, or benefits of a therapy.

As referred herein, “body motion monitoring system” is a system that captures the three-dimensional position, orientation, and a movement of a living body or part of the living body.

In an embodiment, the body motion monitoring system can be a portable and wearable solution.

As referred herein, “brain wave monitoring system” is a system that monitors and measures brain electrical activity, and solutions measuring aspects related to brain function such as sleep patterns, gait, cognition, voice acoustics, and gaze analysis.

In an embodiment, the brain wave monitoring system can be a portable and wearable solution.

As referred herein, “care provider” represents a doctor, a clinician, a nurse, or an attendant who is attending or monitoring a patient.

The term “computing system” encompasses all apparatus, devices, and machines for processing data, including by way of example, a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal (e.g., a machine-generated electrical, optical, or electromagnetic signal) that is generated to encode information for transmission to a suitable receiver apparatus.

As referred herein, “control unit” is an embedded system in an object that controls one or more of the electrical systems, computing systems, electronic systems, or subsystems. In an embodiment the control unit can interact with an external control unit.

The term “communication module” is a module that facilitates communication, that is, it enables transmission and receiving of data from the input and output interfaces to the processor. It also enables communication between the peripheral devices connected with the processor like display, camera, remote servers, and databases. A communication module may be a wired connection between the components or a wireless communication module.

The term “cyber security” as used herein refers to application of technologies, processes, and controls to protect systems, networks, programs, devices, and data from cyber-attacks.

The term “cyber security module” as used herein refers to a module comprising application of technologies, processes, and controls to protect systems, networks, programs, devices and data from cyber-attacks and threats. It aims to reduce the risk of cyber-attacks and protect against the unauthorized exploitation of systems, networks, and technologies. It includes, but is not limited to, critical infrastructure security, application security, network security, cloud security, Internet of Things (IoT) security.

The term “encrypt” used herein refers to securing digital data using one or more mathematical techniques, along with a password or “key” used to decrypt the information. It refers to converting information or data into a code, especially to prevent unauthorized access. It may also refer to concealing information or data by converting it into a code. It may also be referred to as cipher, code, encipher, encode. A simple example is representing alphabets with numbers—say, ‘A’ is ‘01’, ‘B’ is ‘02’, and so on. For example, a message like “HELLO” will be encrypted as “0805121215,” and this value will be transmitted over the network to the recipient(s).

The term “decrypt” used herein refers to the process of converting an encrypted message back to its original format. It is generally a reverse process of encryption. It decodes the encrypted information so that an authorized user can only decrypt the data because decryption requires a secret key or password. This term could be used to describe a method of unencrypting the data manually or unencrypting the data using the proper codes or keys.

The term “cyber security threat” used herein refers to any possible malicious attack that seeks to unlawfully access data, disrupt digital operations, or damage information. A malicious act includes but is not limited to damage data, steal data, or disrupt digital life in general. Cyber threats include, but are not limited to, malware, spyware, phishing attacks, ransomware, zero-day exploits, trojans, advanced persistent threats, wiper attacks, data manipulation, data destruction, rogue software, malvertising, unpatched software, computer viruses, man-in-the-middle attack, data breaches, Denial of Service (DoS) attacks, and other attack vectors.

The term “hash value” used herein can be thought of as fingerprints for files. The contents of a file are processed through a cryptographic algorithm, and a unique numerical value—the hash value—is produced that identifies the contents of the file. If the contents are modified in any way, the value of the hash will also change significantly. Example algorithms used to produce hash values: the Message Digest-5 (MD5) algorithm and Secure Hash Algorithm-1 (SHA1).

The term “integrity check” as used herein refers to the checking for accuracy and consistency of system related files, data, etc. It may be performed using checking tools that can detect whether any critical system files have been changed, thus enabling the system administrator to look for unauthorized alteration of the system. For example, data integrity corresponds to the quality of data in the databases and to the level by which users examine data quality, integrity, and reliability. Data integrity checks verify that the data in the database is accurate, and functions as expected within a given application. Data integrity refers to the accuracy and consistency (validity) of data over its lifecycle. Compromised data is of little use to enterprises, not to mention the dangers presented by sensitive data loss.

The term “alarm” as used herein refers to a trigger when a component in a system or system fails or does not perform as expected. System may enter an alarm state when a certain event occurs. An alarm Indication signal is a visual signal to indicate the alarm state. For example, the heart rate is very low, light emitting diode (LED) may glow red alerting that it is beyond the specified limits, and it turns green when the heart rate is within specified limits. Another example could be, when the cyber security threat is detected, a network administrator may be alerted via sound alarm, a message, a glowing LED, a pop-up window, etc. Alarm indication signal may be reported downstream from a detecting device, to prevent adverse situations or cascading effects.

The term “in communication with” as used herein, refers to any coupling, connection, or interaction using electrical signals to exchange information or data, using any system, hardware, software, protocol, or format, regardless of whether the exchange occurs wirelessly or over a wired connection.

As used herein, the term “cryptographic protocol” is also known as security protocol or encryption protocol. It is an abstract or concrete protocol that performs a security-related function and applies cryptographic methods often as sequences of cryptographic primitives. A protocol describes how the algorithms should be used. A sufficiently detailed protocol includes details about data structures and representations, at which point it can be used to implement multiple, interoperable versions of a program. Cryptographic protocols are widely used for secure application-level data transport. A cryptographic protocol usually incorporates at least some of these aspects: key agreement or establishment, entity authentication, symmetric encryption, and message authentication material construction, secured application-level data transport, non-repudiation methods, secret sharing methods, and secure multi-party computation. Hashing algorithms may be used to verify the integrity of data. Secure Socket Layer (SSL) and Transport Layer Security (TLS), the successor to SSL, are cryptographic protocols that may be used by networking switches to secure data communications over a network.

As used herein, the term “perimeter network” refers to a network closest to a router that is not under the enterprise or organization control. Usually, a perimeter network is the final step a packet takes traversing one of your networks on its way to the internet; and conversely the first network encountered by incoming traffic from the Internet. A network perimeter is a secured boundary between the private and locally managed side of a network, often a company's intranet, and the public facing side of a network, often the Internet. The boundary is defined as a perimeter network.

As used herein, the term “network” may include the Internet, a local area network, a wide area network, or combinations thereof. The network may include one or more networks or communication systems, such as the Internet, the telephone system, satellite networks, cable television networks, and various other private and public networks. In addition, the connections may include wired connections (such as wires, cables, fiber optic lines, etc.), wireless connections, or combinations thereof. Furthermore, although not shown, other computers, systems, devices, and networks may also be connected to the network. Network refers to any set of devices or subsystems connected by links joining (directly or indirectly) a set of terminal nodes sharing resources located on or provided by network nodes. The computers use common communication protocols over digital interconnections to communicate with each other. For example, subsystems may comprise the cloud. Cloud refers to servers that are accessed over the Internet, and the software and databases that run on those servers. Cloud servers are located in data centers all over the world. By using cloud computing, users and companies don't have to manage physical servers themselves or run software applications on their own machines.

As used herein, the term “system hardening” is a collection of tools, techniques, and best practices to reduce vulnerability in technology applications, systems, infrastructure, firmware, and other areas. The goal of system hardening may be to reduce security risk by eliminating potential attack vectors and condensing the system's attack surface.

As used herein, the term “SHA256” stands for Secure Hash Algorithm 256-bit is a hash function and it is used for cryptographic security. Cryptographic hash algorithms produce irreversible and unique hashes. The larger the number of possible hashes, the smaller the chance that two values will create the same hash.

As referred herein, “drug delivery system” is a system that controls a rate at which a drug is released. The drug delivery system also comprises a system that controls a location in the body where it is released. Some systems can control both.

As referred herein, “enclosure” is an area surrounded by a fence or protected by an umbrella or other structure to be kept separate from other areas.

As referred herein, “eye mask” is a covering or blindfold tied to one's head to cover the eyes to block the wearer's sight.

As referred herein, “eye monitoring system” is a device that tracks an eye activity. The term Eye activity as used herein refers to a point of gaze (where one is looking), a motion of an eye relative to the head, eye position, size of pupil, pupil dilation or constriction, blinking patterns, visual attention etc.

As referred herein, “facial expression recognizing system” uses biometric markers to detect emotions in human faces and gesture capture. More precisely, this technology is a sentiment analysis tool and can automatically detect basic or universal expressions like happiness, sadness, anger, surprise, fear, and disgust.

The term “gesture” as used herein refers to a user action that expresses an intended idea, action, meaning, result, and/or outcome. The user action can include manipulating a device (e.g., opening or closing a device, changing a device orientation, moving a trackball or wheel, etc.), movement of a body part in relation to the device or in relation to a reference point, movement of an implement or tool in relation to the device, audio inputs, etc. A gesture may be made on a device (such as on the screen) or with the device to interact with the device.

The term “gesture capture” as used herein refers to a sense or otherwise a detection of an instance and/or type of user gesture. The gesture capture can be received by sensors in three-dimensional space. Further, the gesture capture can occur in one or more areas of a screen, for example, on a touch-sensitive display or a gesture capture region. A gesture region can be on the display, where it may be referred to as a touch sensitive display, or off the display, where it may be referred to as a gesture capture area.

As referred herein, “armrest” is a part of the article of furniture that supports the arm.

As referred herein, “bridge” is a surface that is juxtaposed to an enclosure attached to an article of furniture.

As referred herein, “handlebar” is a handgrip to support moving a device from one place to another.

As referred herein, “heat therapy” or “thermotherapy” is the use of heat in therapy, such as for pain relief, rehabilitation purposes, and health. The heat therapy can be done using dry heat and moist heat. Both types of heat therapy should aim for “warm” as the ideal temperature instead of “hot.” It can take the form of a hot cloth, hot water bog, ultrasound, heating pad, hydrocollator packs, whirlpool baths, cordless FIR heat therapy wraps, and others.

As referred herein, “in-built” or “built-in” means forming an integral part of a structure or object.

As referred herein, “light emitting device” is a device that emits light when an electric current passes through it. In an embodiment, the light therapy device is programmed to emit light based on an input wavelength, of an input colour for an input time. The light emitting device is height and direction adjustable.

As referred herein, “light therapy” is a way to treat psychological or physiological disorders by exposure to artificial light.

As referred herein, “massage therapy” is a type of integrative medicine wherein the soft tissues of the body are manipulated using varying degrees of pressure and movement. Massage involves acting on and manipulating the body with pressure—structured, unstructured, stationary, or moving—tension, motion, or vibration, done manually or with mechanical aids.

As referred herein, “magnetic therapy” is a process of treating a physical, physiological, or psychological problem using a weak static magnetic field. The magnetic field can be produced by a permanent magnet or by an electrically powered device. The magnetic therapy also comprises transcranial magnetic stimulation. It also comprises the medicine practice of electromagnetic therapy, which uses a magnetic field generated by an electrically powered device.

As referred herein, “sound therapy” refers to a range of therapies in which sound is used to treat physical and mental conditions. One of these therapies is music therapy, which can involve a person listening to music for conditions such as stress and muscle tension.

As referred herein, “induction charger” is a device that uses electromagnetic waves to transfer energy and charge devices wirelessly.

As referred herein, “living body” pertains to an animal or a person.

In an embodiment, the living body is a patient. The term patient or subject as used herein refers to a person receiving or registered to receive medical treatment. a patient is also referred to as a user as he receives treatment using the therapy system.

As referred herein, “physiological state” is a condition or state of the body, bodily functions, mental state, and emotional state of a living being. Examples include but not limited to asphyxia, consciousness, alertness, acapnia, hypercapnia, hypothermia, hyperthermia, upset, cryptobiosis, good health, myasthenia, atherosclerosis, myocardial infarction, angina pectoris, arrhythmias (irregular heartbeat), chronic heart failure, blood pressure, glucose or blood sugar, temperature, drowsiness, hallucinations, slow breathing, dry mouth, anxiety, vomiting, confusion, drowsiness, slurred speech, rambling speech, lack of coordination, mood changes, involuntary eye movement, dizziness, alertness, restlessness, dilated pupils, nasal congestion, behavior changes, chills, sweating, loss of memory, teeth clenching, coordination problems, impulsive behavior, pain sensitivity, tremors, rashes, euphoria, sense of pain, etc. Some signs may be directly measured, for example, heart rate and some may be indirectly measured, for example, measuring brain activity to analyze the state of consciousness. In the embodiments herein, the term “physiological” is intended to be used broadly, covering both physical and psychological characteristics of or from the body of an organism.

As referred herein, “power module” is a module that provides power to the device and the components of the device.

As referred herein, “psychedelic drug” is psychotomimetic drug or hallucinogen, or any of the so-called mind-expanding drugs that are able to induce states of altered perception and thought, frequently with heightened awareness of sensory input but with diminished control over what is being experienced.

As referred herein, “audio device” or “sound emitting device” is an output device capable of generating a frequency corresponding to a normally audible sound wave.

As referred herein, “support surface” is a brace, pillow, or mattress on which part or the full body rests. Static support surfaces are made or filled with materials such as air, fabric, foam, or gels. Dynamic support surfaces are filled with moving air, beads, or fluid that circulates by electromechanical energy.

As referred herein, “storage space” is a place or an area designed for holding or safe-keeping objects.

As referred herein, “sensor” is a device, module, machine, or subsystem whose purpose is to detect physiological or biometric changes and send the information to other electronics, frequently a computer processor. The sensor is used with other electronics and it enables recording, presentation or response to such detection or measurement using processor and optionally memory. A sensor and processor can take one form of information and convert such information into another form, typically having more usefulness than the original form. For example, a sensor may collect raw physiological or environmental data from various sensors and process this data into a meaningful assessment, such as pulse rate, blood pressure, or air quality using a processor. A “sensor” herein can also collect or harvest acoustical data for biometric analysis (by a processor) or for digital or analog voice communications. A “sensor” can include any one or more of a physiological sensor (e.g., blood pressure, heart beat, etc.), a biometric sensor (e.g., a heart signature, a fingerprint, etc.), an environmental sensor (e.g., temperature, particles, chemistry, etc.), a neurological sensor (e.g., brainwaves, EEG, etc.), or an acoustic sensor (e.g., sound pressure level, voice recognition, sound recognition, etc.) among others. A variety of microprocessors or other processors may be used herein. Although a single processor or sensor may be represented in the figures, it should be understood that the various processing and sensing functions can be performed by a number of processors and sensors operating cooperatively or a single processor and sensor arrangement that includes transceivers and numerous other functions as further described herein.

As referred herein, “radio frequency identification” or “RFID” is a form of wireless communication that uses low-power radio frequency waves to receive and transmit data.

As referred herein, “visual display” is a device capable of generating an image or video on a device. The device may comprise a terminal in which a cathode ray tube, liquid-crystal, or plasma display device is used for the visual presentation of data.

As referred herein, “vital monitoring system” provides standard data on body temperature, pulse rate, respirations, and blood pressure. It can also provide data on ECG, pulse oximetry (SPO₂), end tidal carbon dioxide (EtCo₂), cardiac output, and agent analysis.

As referred herein, “electrocardiography” is the process of producing an electrocardiogram (ECG or EKG), a recording of the heart's electrical activity.

As referred herein, “rapid eye movement sleep” (REM sleep or REMS) is a unique phase of sleep in mammals and birds, characterized by random rapid movement of the eyes, accompanied by low muscle tone throughout the body, and the propensity of the sleeper to dream vividly.

As referred herein, a “DC motor” is any of a class of rotary electrical motors that converts direct current (DC) electrical energy into mechanical energy.

As referred herein, “UV light” or “Ultraviolet (UV) light” is a form of electromagnetic radiation with wavelength from 10 nm (with a corresponding frequency around 30 PHz) to 400 nm (750 THz), shorter than that of visible light, but longer than X-rays.

As referred herein, “real-time locating systems” (RTLS), also known as real-time tracking systems, are used to automatically identify and track the location of objects or people in real time, usually within a building or other contained area. Wireless RTLS tags are attached to objects or worn by people, and in most RTLS, fixed reference points receive wireless signals from tags to determine their location.

As referred herein, a “SIM” or “SIM card” (full form Subscriber Identity Module or Subscriber Identification Module), is an integrated circuit intended to securely store the international mobile subscriber identity (IMSI) number and its related key, which are used to identify and authenticate subscribers on mobile telephony devices (such as mobile phones and computers). It is also possible to store contact information on many SIM cards.

In an aspect a device is described herein. The device comprises a drug delivery system; a support surface to support at least a portion of a living body; an enclosure to surround at least the portion of the living body; a bio-monitoring system to monitor a physiological state of the living body. In an embodiment, the device comprises an article of furniture.

In an embodiment, the article of furniture is one of a chair, a reclining chair, a bed, a stretcher, or a chair cum bed.

In an embodiment, the device is a reclining chair (hereafter chair).

In an embodiment, the living body comprises a human body or an animal body.

In an embodiment, the blanket is at least one of a weighted blanket, a grounding mat, a magnetic therapy pad and a temperature adjustable blanket or combination thereof. The weighted blanket provides a deep pressure stimulation. The deep pressure stimulation can help reduce autonomic arousal. This arousal is responsible for many of the physical symptoms of anxiety, such as increased heart rate. The added pressure may help to calm heart rate and breathing. They are helpful in treatment of anxiety, insomnia, and sleep disorders, to improve attention and reduce hyperactive movements. The grounding mat creates an electrical connection between the living body and the earth. This connection allows electrons to flow from the earth and into the living body to create a neutral electrical charge. The grounding mats usually connect via a wire to the ground port of an electrical outlet. The magnetic therapy pad may provide a static magnetic field via a plurality of magnets or an electromagnetic field.

In an embodiment, the bio-monitoring system comprises a vital monitoring system. In another embodiment, the vital monitoring system comprises at least one of an EEG system, an ultrasound transducer, a heart rate monitor, a breathing monitor, and a pulse rate monitor. In yet another embodiment, the bio-monitoring system further comprises at least one of a body motion monitoring system, a brain wave monitoring system, a facial expression recognizing system and an eye monitoring system. In yet another embodiment, the bio-monitoring system comprises a biofeedback control system and wherein an output from the biofeedback control system controls a delivery of a drug by the drug delivery system. In yet another embodiment, the biofeedback control system controls other components of the device related to a therapy.

In an embodiment, the blanket comprise one or more physiological sensors connected to bio-monitoring system to predict when a person will have a hot flash and to proactively provide localized cooling or accelerated airflow for that person for a limited time to alleviate the adverse effects of that hot flash. In an example, a physiological sensor can be a body temperature sensor, skin conductance sensor, or EEG sensor.

In an embodiment, the device further comprises an armrest, wherein the armrest is foldable. The armrest is designed in such a way that it can be rotated, adjusted and/or removed on a need basis. In an embodiment, the height or angle of the armrest is adjustable. In an embodiment, the foldable armrest is provided with a heating pad. In another embodiment, the armrest is provided with a cooling pad. In yet another embodiment, the armrest is provided with a temperature adjustable pad.

In an embodiment, the device further comprises wheels. In another embodiment, the wheels are provided with wheel stoppers. In yet another embodiment, the wheel is equipped with a double caster wheel mechanism to minimize overall height and maximize load capacity for the chair.

In an embodiment, the device further comprises a handlebar. In another embodiment, the handlebar is covered with a grip wrap.

In an embodiment, the device further comprises one or more storage spaces. In another embodiment, one of the storage spaces acts as a sterilization unit. In yet another embodiment, the sterilization unit uses a wavelength-based sterilization technique in the sterilization unit for sterilization of accessories.

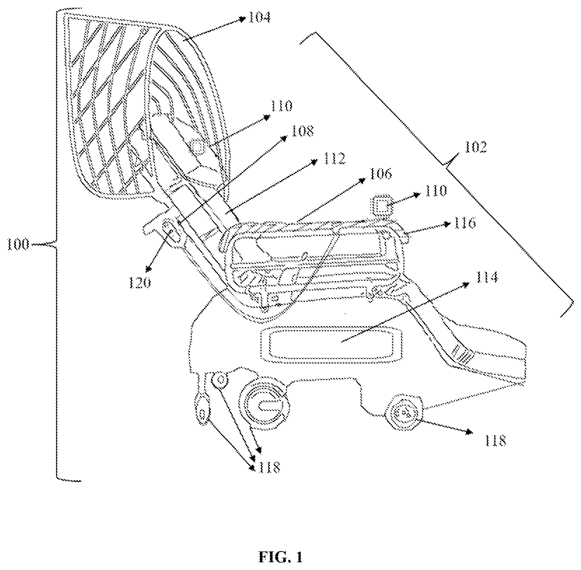

FIG. 1 depicts the device comprising a chair, in one or more embodiments. The chair 100 comprises a support surface 102, an enclosure 104, a biomonitoring system 110 and a drug delivery system 108. The chair further comprises a size adjustable armrest 106, one or more storage space 114 and a plurality of wheels 118. The chair can be adjusted to form a bed. The support surface comprises a blanket 112 and the armrest 106 comprises a temperature adjustable pad 116. The drug delivery system 108 comprises a biofeedback-control unit 120 to control a drug delivery.

In an embodiment, the support surface further comprises a blanket. The device further comprises a storage space 114 and a plurality of wheels 118.

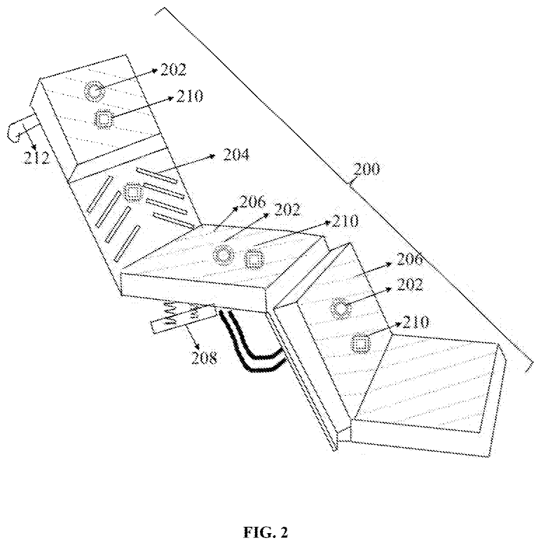

FIG. 2 depicts a support surface of the chair comprising a blanket, in one or more embodiments. The support surface 200 comprises a blanket 206. In an example, speakers, vibration elements 204, and a biomonitoring device. The support surface 200 is provided with spring support 208 and a handlebar 212.

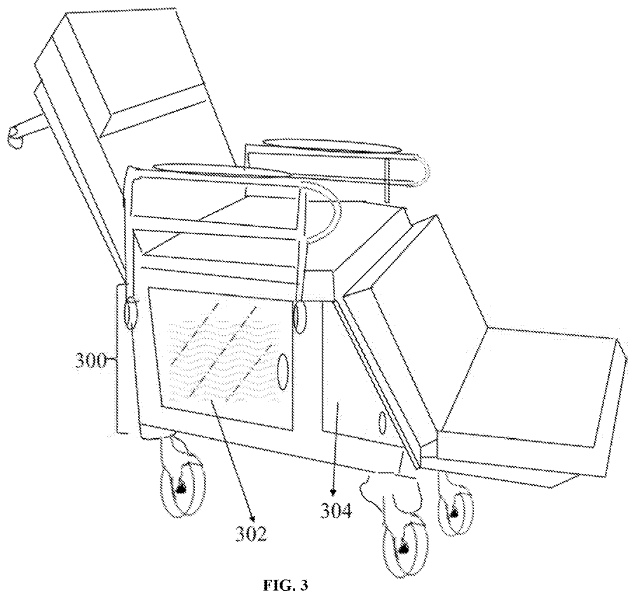

FIG. 3 depicts a storage space of the chair, in one or more embodiments. The storage space 300 comprises a wave-based sterilization unit 302 and a personal cubby 304 to store personal items.

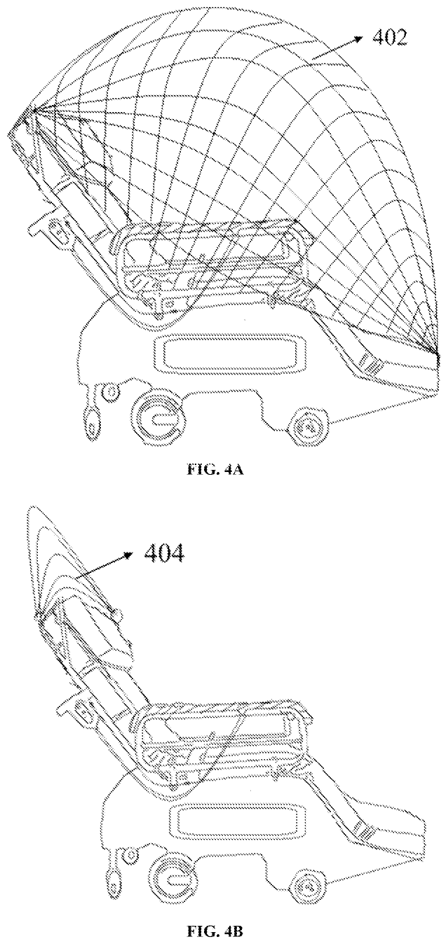

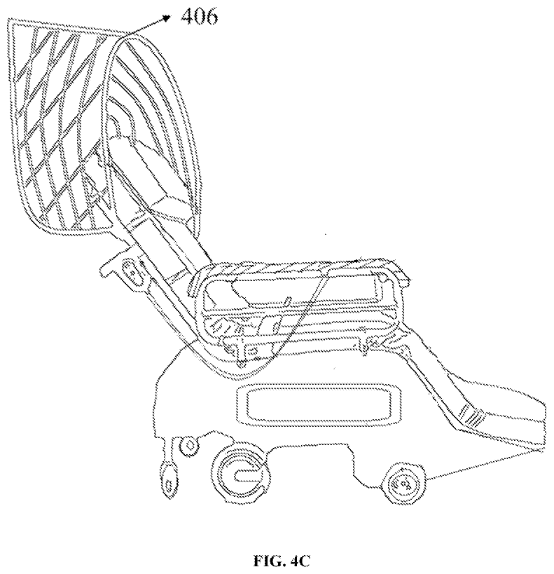

In an embodiment, the enclosure comprises an adjustable dome shaped housing that allows freedom of movement. FIG. 4A depicts a closed enclosure of the chair, in one or more embodiments. In an example, the enclosure 402 covers the full support surface. FIG. 4B depicts an open enclosure of the chair, in one or more embodiments. In an example, the enclosure 404 does not cover any portion of the support surface. FIG. 4C depicts a partially open enclosure of the chair, in one or more embodiments. In an example, the enclosure 406 covers a part of the support surface. In another embodiment, the enclosure is detachable.

In an embodiment, the adjustable dome shaped housing further comprises a bridge, wherein the bridge is foldable. In yet another embodiment, the height of the bridge is adjustable.

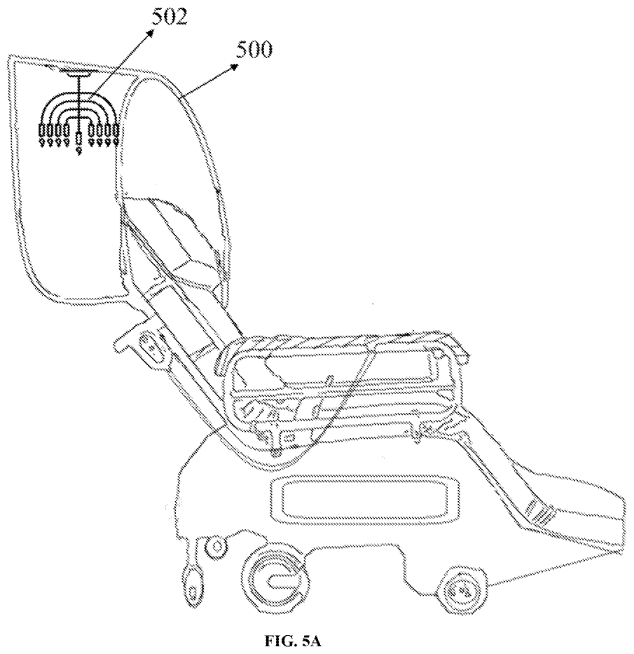

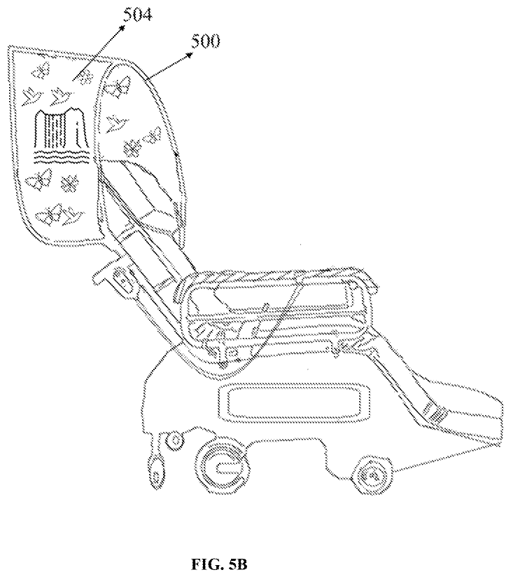

In an embodiment, the bridge comprises a light emitting device. FIG. 5A depicts the bridge of the chair comprising a light emitting device, in one or more embodiments. In an example, the bridge 500 comprises a light emitting diode (LED) panel 502 that emits light of a wavelength suitable for the therapy. The light can be a chromatic light or an achromatic light. In another example, the bridge comprises micro-LEDs embedded in the bridge.

In an embodiment, the bridge comprises an optical surface. In yet another embodiment, the bridge comprises a visual display. FIG. 5B depicts the bridge of the chair comprising a visual display, in one or more embodiments. In an example, the bridge 500 comprises a foldable display 504. In another example, the foldable displays use organic light emitting diode (OLED) technology. The foldable display may comprise OLED panels embedded in a flexible plastic substrate. When electricity is passed from cathode to anode, the organic layers between them release energy in the form of light to display an image, a video, or a dynamic pattern. In another example, the visual display is projector based. The projector can be one of a digital light processing (DLP) projector, a liquid crystal display (LCD) projector, and a light emitting diode (LED) projector.

In an embodiment, the device can be adjusted to an upright position, a reclined position, and a horizontal position.

In an embodiment, the chair is equipped with a remotely managed interface for remote adjustment of the chair position and chair height through a cloud-based android application. In another embodiment, the chair is voice actuated. The control unit comprises a voice recognition module to detect a keyword and perform an action such as inclination, reclination, height adjustment, turning on or off a component of the chair.

The device is equipped with a system for adjustment of the chair into bed and vice-versa. The automatic adjustment of the chair into bed can be done using one of a slider mechanism, a scissor lifter mechanism, a fork lifter mechanism, and a lifting mechanism.

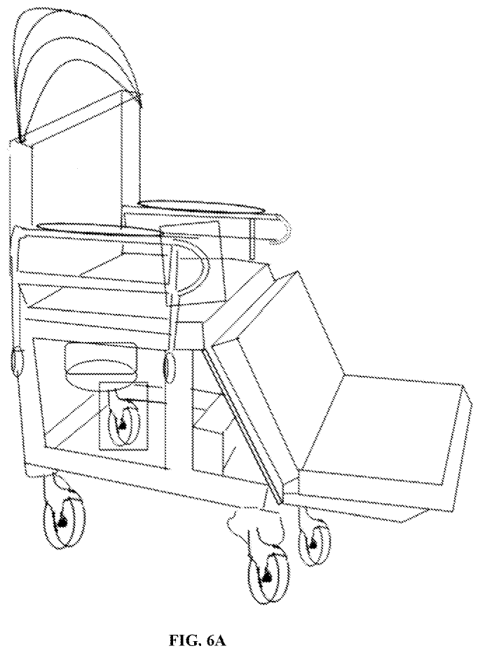

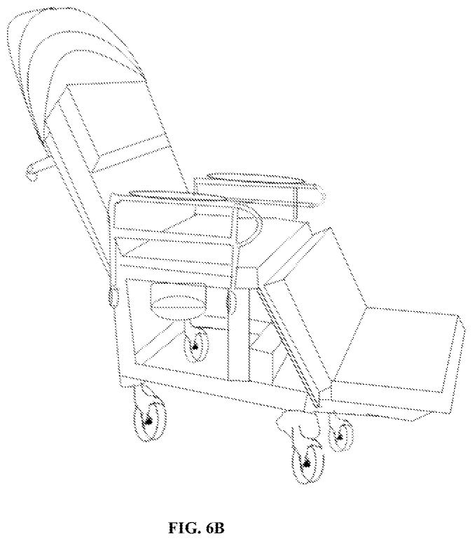

In an embodiment, a hydraulic scissor lifter can be used to convert the chair into a bed. In an embodiment, the chair is joystick controllable and can be converted to a bed using the lifting mechanism. A DC motor is integrated to the chair for automatic switching of the chair into a bed and vice-versa. The chair can be operated with hydraulics for adjustment of height of the chair according to convenience. FIG. 6A depicts the chair adjusted in an upright position and FIG. 6B depicts a chair adjusted in a reclined position, in one or more embodiments.

In an embodiment, the device further comprises an attachable device. In another embodiment, the attachable device comprises at least one of a sound emitting device, a light emitting device, an aroma infusion device, an eye mask, and a computing system. In yet another embodiment, the attachable device and the bio-monitoring system can be remotely controlled via the computing unit. In yet another embodiment, the attachable device is connected to the device through one of a wireless and a wired connection.

In an embodiment, the sound emitting device is one of an ear pod, a 360-degree sound speaker and a headphone. In another embodiment, the sound emitting device is operable to provide one of a programmed meditation audio, a brain stimulating wave frequency, music, and a nature sound. The transmitted audio and the brain stimulating wave frequency may include translated frequencies. These translated frequencies are generated by a translation of higher frequencies, which can mainly be heard, to lower frequencies, which can mainly be felt.

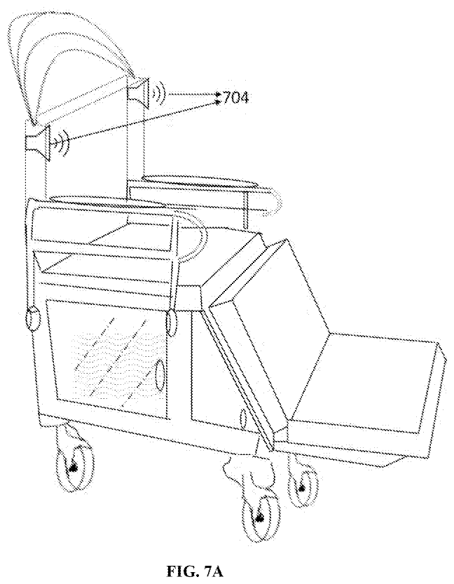

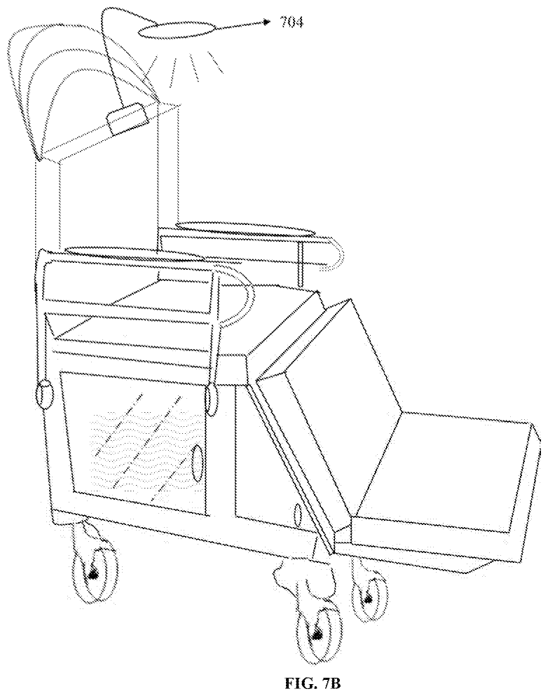

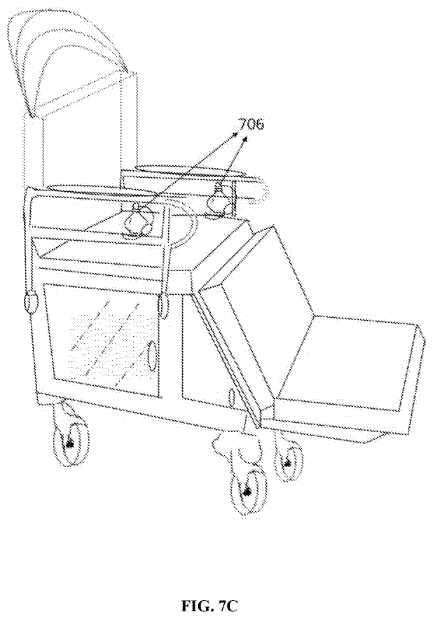

In an embodiment, the sound emitting device is disposed within the support surface and is surrounded by the blanket and a surrounding foam. In another embodiment, the sound emitting device is attached to the eye mask. In yet another embodiment, the sound emitting device is attached to a head resting portion of the device. The support surface comprises covering layers that are designed to be very compressible to conform to the living body's head or back for comfort purposes and to allow sound and vibration energy to pass with minimal attenuation and obstruction. FIG. 7A depicts a sound emitting device as an attachable device of the chair, in one or more embodiments. In an example, the device comprises a plurality of 360-degree sound speakers 704.

In an embodiment, the light emitting device provides a predetermined light frequency for a predetermined duration. In at least one of the various embodiments, the living body is positioned at a particular location in the enclosure that includes a light emitting device located at one or more of the supporting surfaces, the bridge, or one or more walls of the enclosure. Each of the plurality of light sources is arranged to emit light towards the particular location over one or more periods of time based on one or more selected settings for duration, intensity, ramp up, ramp down, plateau, oscillation, and the like. In at least one of the various embodiments, the light is between 290 nm and 900 nm. In at least some of the various embodiments, the light is a UV light, a blue light, or an infrared light. The supporting surface, the ceiling, and each wall of the enclosure is configured to focus light at the location when the light is emitted by the plurality of light emitting devices so that the living body is bathed in the light. For example, one or more lenses may be employed to focus the light emitted by the light emitting device at a particular location in the enclosure. In an embodiment, the device further comprises an eye mask attachment. FIG. 7B depicts a light emitting device as an attachable device of the chair, in one or more embodiments. In an example, the device comprises the light emitting device 702.

In an embodiment, the attachable device is an aroma infusion device. In an example the aroma infusion device is an electric aroma infusion device. The aroma infusion device can emit a single aroma or a combination of aromas. The aroma infusion device can also be programmed to infuse a specific kind of aroma for a specific time interval.

FIG. 7C depicts an aroma emitting device as an attachable device of the chair, in one or more embodiments. In an example, the aroma infusion device is attached to the armrest. The aroma infusion device may also be embedded in the support surface. In an example, the device comprises a plurality of aroma infusion devices 706.

In an embodiment, the device further comprises a computing system. In another embodiment, the computing system is configured to communicate with the drug delivery system, the biofeedback monitoring system, and the attachable device. In yet another embodiment, the computing system can take and execute a control instruction from a remote location.

In yet another embodiment, the computing system is at least one of a smartphone, a tablet, a laptop, a desktop, and a built-in computer.

In an embodiment, the chair is equipped with a remotely managed interface monitoring the physiological state of the person through a cloud-based android application.

In yet another embodiment, the device further comprises a control unit, wherein the control unit comprises an input module, a processor, a communication module, a database, a universal serial bus, a controller, a display, and a power module and wherein the control unit can remotely take and execute a control instruction.

FIG. 8 depicts a control unit of the device, in one or more embodiments. The control unit 800 of the device comprises a processor 802, a database 804, a communication module 806, an input module 808, a display 810, a power module 812, a controller 814, and a universal serial bus (USB) 816. The controller 814 is configured to enable feedback control of a therapy and pulse control circuit for timer control.

The control unit may also comprise a memory. Memory is a computer-readable memory, such as a read-only memory (ROM), random-access memory (RAM), a flash memory, magnetic media memory, and/or other memory for storing data to be used by and/or generated by the device and/or executable program code that may be executed by the data processor.

In FIG. 8, the control unit 800 further comprises a cyber security module 828 and the communication module 806 that communicates to a server 820 via a network 818 using the cyber security module 828.

In an embodiment, the device can communicate data to a server via the communication module. In yet another embodiment, the control unit comprises a cyber security module.

In an embodiment, the cyber security module further comprises an information security management module providing isolation between the system and the server. In yet another embodiment, the information security management module is operable to: receive data from at least one of the input module, the bio-monitoring system, the attachable device, the drug delivery system, and the database; exchange a security key at a start of the communication between the communication module and the server; receive the security key from the server; authenticate an identity of the server by verifying the security key; analyze the security key for a potential cyber security threat; negotiate an encryption key between the communication module and the server; encrypt the data; and transmit the encrypted data to the server when no cyber security threat is detected. In an embodiment, the integrity check is a hash-signature verification using a SHA256 or similar method.

In an embodiment, the information security management module is operable to: exchange a security key at a start of the communication between the communication module and the server; receive the security key from the server; authenticate an identity of the server by verifying the security key; analyze the security key for a potential cyber security threat; negotiate an encryption key between the system and the server; receive encrypted data; decrypt the encrypted data; perform an integrity check of the decrypted data; and transmit the decrypted data to at least one of the input module, the bio-monitoring system, the drug delivery system, and the database through the communication module when no cyber security threat is detected. In yet another embodiment, the information security management module is configured to raise an alarm when the cyber security threat is detected. In yet another embodiment, the information security management module is configured to discard the encrypted data received if the integrity check of the encrypted data fails. In yet another embodiment, the information security management module is configured to check the integrity of the encrypted data by checking accuracy, consistency, and any possible data loss during the communication through the communication module. In yet another embodiment, the information security management module is configured to perform asynchronous authentication and validation of the communication between the communication module and the server. In yet another embodiment, wherein a perimeter network provides an extra layer of protection. In yet another embodiment, wherein the perimeter network protects the system from a cyber security threat by using a plurality of firewalls.

According to an embodiment, the device provides the display 810 to show a present state of the device and enables a care provider to define new rules and modify or delete existing rules of an operation. According to an embodiment, the device provides an interface for receiving input for creating and/or modifying rules from computing systems and/or various instruments for monitoring the physiological state of a person. In another embodiment, there can be one or more knobs on the device, for controlling operations of electrical components in the device.

In an embodiment, the control unit stores instructions, and the processor executes the instructions to perform actions. For example, the actions may include generating a plurality of therapy settings based on one or more of: measured information from the living body, such as health-disease spectrum values or physical attributes, reported living body data, care provider or operator observation information, such as observations regarding the stress level of the living body, previous therapy settings of the living body, heuristic information regarding the living body or a plurality of other living bodies who have received therapy, or operator selected information; selecting one or more additional settings to provide therapy to a living body; positioning the living body within an enclosure to receive therapy; collecting post-therapy information regarding the living body; and generating a report regarding the living body based on the post-therapy information. The display may present a report to the operator and/or the care provider.

According to some embodiments, the processor 802 is coupled with appropriate user interface peripherals. Several types of computing systems may be used, such as a personal computer system, a laptop computer system, a handheld computer system, or the like.

In an embodiment, the device settings for a patient are stored and retrieved for future automatic setting of the device. All the person (patient) specific data is kept private and secured. Other data such as data analytics and data aggregations are to be anonymized.

In an embodiment, the power module 812 further comprises one or more of a rechargeable battery, a non-rechargeable battery, a solar cell, a chemical reaction power generator, a power input port that connects to an external power line, or any other device configured to provide power to components of the device.

In an embodiment, the power module is arranged and disposed to provide wireless charging through an induction charger. A sensor may sense when no one is in a room in which the chair is located and may start battery charging when no one is in the room. Upon sensing a person entering the space, the device may automatically stop charging the battery. By charging the battery only when no one is in the room, any concerns a person may have about being present within an inductive field are avoided. As another instance, when no person is located near the charging mat (e.g., a specific space), the system may start the charging process. In at least some embodiments, it is contemplated that some type of indicator may be provided that affirmatively indicates whether a chair battery is being charged. For example, there may be an indicator that signals to a user of a chair that the chair battery is not being charged while the chair is in use. Where an indicator of no charge is provided, any user concern regarding simultaneous charging may be eliminated. For example, in some cases a small device may be provided for placement on the top surface of a workstation table or the like that includes red and green LED indicators aligned with a two-state legend that indicates “charging” and “not charging,” respectively. Here, when the green LED is illuminated, a chair user would know that the battery is not being charged and when the red LED is illuminated a person could determine that the chair is currently being charged.

The chair is made with a low weight and high strength material. In an embodiment, the chair components are made of premium cold rolled steel with electrostatic powder coating. The covering layer can be single layered or multi-layered made of highly porous material, foam, or a fiber.

In an embodiment, the device has an autonomous navigation module to navigate autonomously on a predefined track. The device can move autonomously and can navigate through a point O1 to a point O2 based on input provided by the living body. The mobility can be managed by a care provider, an intelligent control system or the living body positioned on the device. In another embodiment, the mobility access is limited for the living body receiving treatment.

The device has various functions such as autonomous navigation, obstacle avoidance, human-machine dialogue, and provision of special services. The device combines various technologies in the robotics field, for example, robot navigation and positioning, machine vision, pattern recognition, multi-sensor information fusion, and human-machine interaction.

The device may use an intelligent robot to implement various functions, such as moving, changing directions, stopping, sensing the surroundings, drawing a map, or planning a route. The device may use a motion sensor, a motion assembly, and a positioning system to navigate autonomously through a defined path. The motion sensor comprises at least one of an accelerometer, a gyroscope, a sonar, an infrared distance sensor, an optical flow sensor, a laser radar, and a navigation sensor. The motion assembly comprises a motion unit and a carrier. The motion unit may include a plurality of wheels. The carrier may be configured to hold the sonar or the optical flow sensor to detect a device or an obstacle. In some embodiments, the carrier may include the accelerometer and the gyroscope to keep balance of the motion assembly. In some embodiments, the carrier may include various sensors, such as the infrared distance sensor, to obtain information on location of the device and distance between the device and an obstacle. The positioning system comprises at least one of a global positioning system (GPS), a global navigation satellite system (GLONASS), a compass navigation system (COMPASS), a Galileo positioning system, a quasi-zenith satellite system (QZSS), a wireless fidelity (Wi-Fi) positioning technology, or the like, or any combination thereof. An algorithm, decision logic schemes, or Al can use the available sensor data for deriving a proper way to maneuver the device through said environment.

In an embodiment, the device may use a radio frequency identification (RFID) technology for device location management and real-time location systems (RTLS systems) to track a movement of the device. In an embodiment, the device comprises an active RFID tag. Active RFID uses battery-powered tags that advertise their identity to various access points or readers. These access points often then transfer the location of each tagged item to a gateway. In an example, AirFinder active RFID tags calculate their location relative to reference points and send this data to nearby readers. The readers then send the location data to the gateway, which is then sent to the AirFinder web application. The application takes the data and provides the care provider with an estimated location of the tagged device. In another embodiment, the device comprises an intelligent RFID tag. In an example, intelligent active RFID tags wake up at established intervals, scanning their environment for nearby fixed reference point beacons, which they use to calculate their locations. Then the tags send this data back to the reader. The data transmitted through the backend are device location and condition changes. The reduced data burden greatly increases the tag's battery life.

In an example, a care provider may grant a patient access to chair movement as the patient receives their treatment. In an example, the treatment is chemotherapy. Through an application programming interface, the nurse may turn on a radar path system for the device to follow. In an example, the radar path systems are located within device ceiling or floor tiles which may guide the chair towards the treatment room, elevator, common rooms, outdoor garden, etc. The device movement can be monitored from the application programming interface through a navigation tracking system. Within the application programming interface, the nurse may summon the device back to the treatment room remotely. The device includes long-range radar, short-range radar, and has the ability to monitor its surroundings through a 360-camera monitor. The device will communicate with the living body before it makes any movement and provide an audio notification before moving.

The device is connected to another smart electronic device/s and/or the internet using, but not limited to, Bluetooth, Wi-Fi, or/and SIM card. In an embodiment, the device gives the user or/and care provider live feedback about user health metrics and the position status using a data representation method.

In an aspect another device is described herein. The device comprises a support surface to support at least a portion of a living body; means for providing a therapeutic stimulus; an enclosure to surround at least the portion of the living body; a bio-monitoring system; and wherein the bio-monitoring system monitors a physiological state of the living body.

In yet another embodiment, the psychedelic drug, the chemotherapy, and the nutrient infusion are provided using a drug delivery system.

In yet another embodiment, the drug delivery system is monitorable and controllable from a remote location.

In yet another embodiment, the audio stimulus is provided using a sound emitting device.

In yet another embodiment, the audio stimulus is one of a programmed meditation audio, a brain stimulating frequency, a music, and a nature sound.

In yet another embodiment, the aroma stimulus is provided using one of a built-in aroma infusion device and an aroma pouch.

In yet another embodiment, the magnetic field is provided via one of a far infrared heat, pulsed electromagnetic field, red light therapy, negative ions, and natural crystals.

In yet another embodiment, the touch stimulus is provided using the support surface.

In yet another embodiment, the light stimulus is provided using a light emitting device.

In yet another embodiment, the enclosure further comprises a bridge, wherein the bridge is foldable.

In yet another embodiment, the device further comprises an eye mask attachment.

In an aspect yet another device is described herein. The device comprises a support surface to support at least a portion of a living body; an enclosure to surround at least the portion of the living body; the enclosure comprising a visual display; and a bio-monitoring system; and wherein the bio-monitoring system monitors a physiological state of the living body.

In an embodiment, the visual display provides a pictorial display containing imagery designed to create relaxation and reduce stress. In another embodiment, the device is configured to provide a therapeutic stimulus.

In an aspect a method is described herein. The method comprises steps of providing a therapy to a living body using a device; monitoring a physiological state of the living body using the device; and estimating an effect of the therapy using a bio-monitoring system; and modifying the therapy based on the change in the physiological state; and wherein the device comprises an article of furniture comprising a support surface to support at least a portion of a living body; an enclosure to surround at least the portion of the living body; and the bio-monitoring system that monitors a change in the physiological state of the living body.

In an embodiment, the therapy is at least one of a psychedelic drug therapy, a chemotherapy, a nutrient infusion therapy, a sound therapy, an aromatherapy, a magnetic therapy, a massage therapy, a pressure therapy, a light therapy, and a visual based stress relief therapy.

Sound has harmonic properties. Harmonics (the sharps above fundamental sound) have the capacity to stimulate the brain (epiphysis) and to produce states of modified consciousness facilitating relaxation (alpha waves). Sound recharges the cortex and drives the nervous system. Sound travels and stimulates different zones of the brain and can balance the two brain hemispheres. Moreover, sonorous vibrations impact the body through resonance with the cellular fluids in the body. Vibrations alternating high and low frequencies, as in the sessions of the present methodology, are designed to improve functions of the mechanism of the ear. Sound allows access to emotions buried in the unconscious and favors the releasing of “emotional crystals” with the spontaneous appearance of images of the past accompanied sometimes by sadness and anger not yet transformed. Additionally, sound brings about relief through its effect on the central nervous system through improving cognitive capabilities (attention, memory) and psycho-motor capabilities through which it is known to reduce the effects of stress.

In another embodiment, the sound therapy is provided using a sound emitting device. In yet another embodiment, the sound emitting device can be remotely controlled to provide an audio stimulus. In yet another embodiment, the audio stimulus is one of a programmed meditation audio, a brain stimulating frequency, a music, and a nature sound.

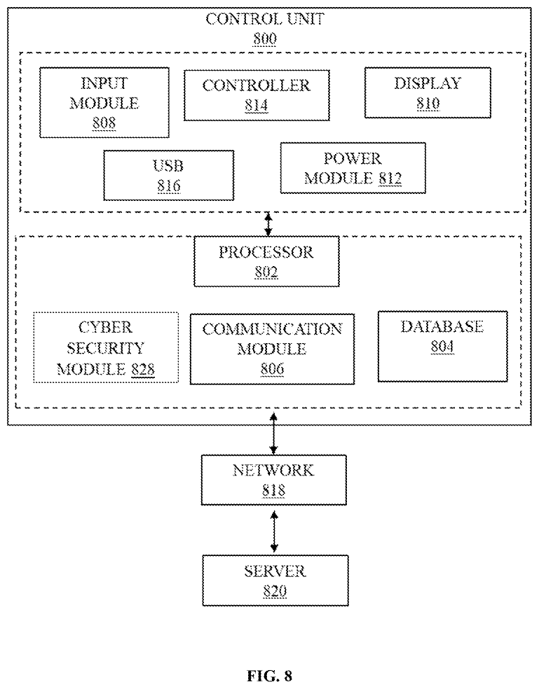

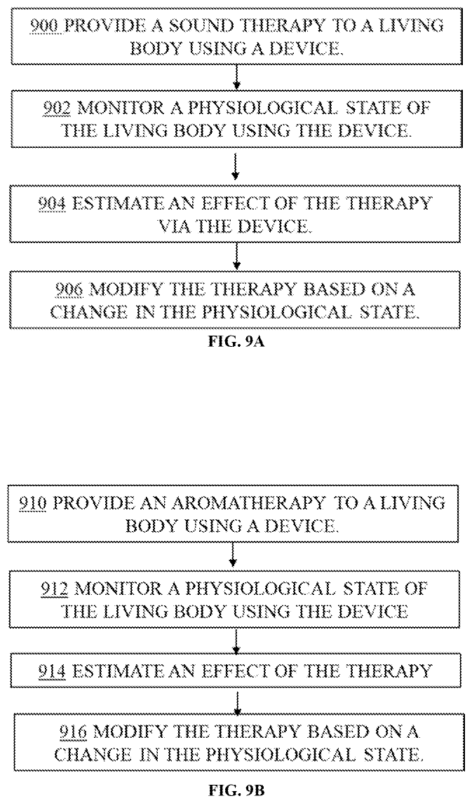

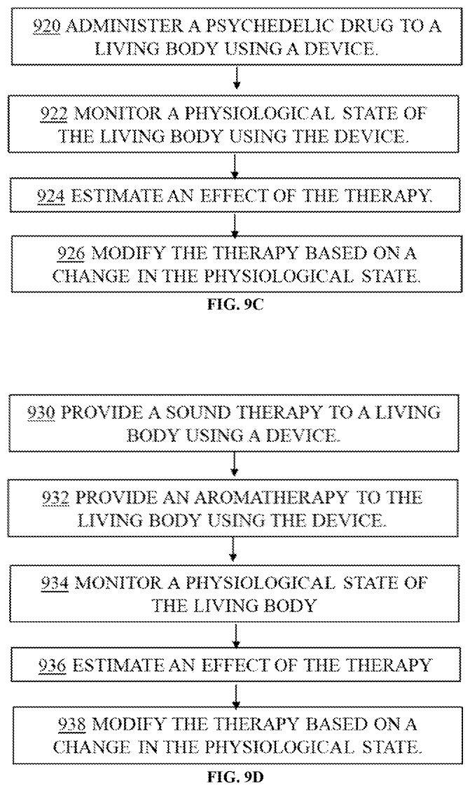

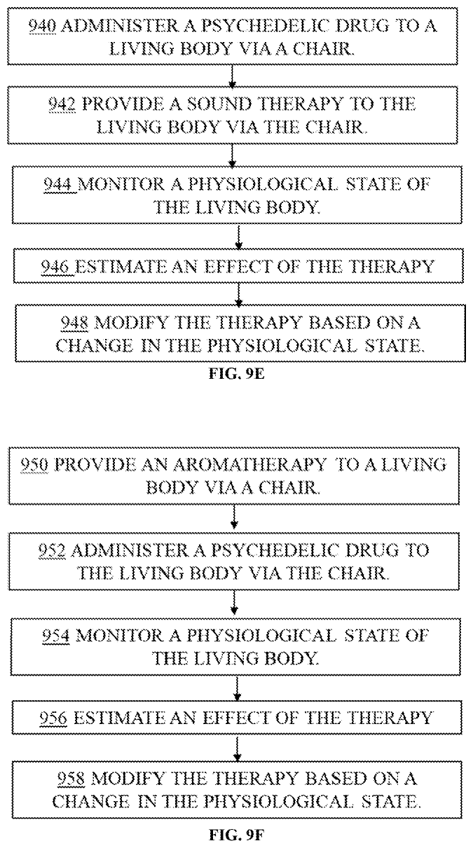

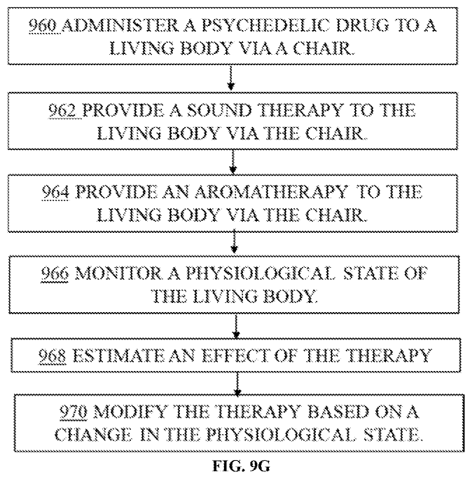

FIG. 9A provides a flow chart of providing a sound therapy and estimating an effect using the device, in one or more embodiments. The method comprises the following steps:
Step 900: Provide a sound therapy to a living body using a device (comprising an article of furniture).
Step 902: Monitor a physiological state of the living body using the device.
Step 904: Estimate an effect of the therapy via the device.
Step 906: Modify the therapy based on a change in the physiological state.

Aromatherapy is also filled with vibrations of an electromagnetic spectrum. Such therapy helps promote the healing of the body as well as activating the brain's emotional centers for peacefulness of the mind. In an embodiment, the aromatherapy is provided using one of a built-in aroma infusion device and an aroma pouch. One or more aromas are dispensed intermittently from the aroma infusion device throughout a treatment session.

FIG. 9B provides a flow chart of providing an aromatherapy and estimating an effect using the device, in one or more embodiments. The method comprises the following steps:
Step 910: Provide aromatherapy to a living body using a device (comprising an article of furniture).
Step 912: Monitor a physiological state of the living body using the device.
Step 914: Estimate an effect of the therapy.
Step 916: Modify the therapy based on a change in the physiological state.

In yet another embodiment, the psychedelic drug therapy is done using a psychedelic drug, wherein the psychedelic drug is one of a ketamine, esketamine, methylenedioxy-methylamphetamine, psilocybin, cannabis, an antidepressant, an anti-anxiety drug, an antipsychotic, and a psychoactive drug.

FIG. 9C provides a flow chart of infusing a psychedelic drug to a person and estimating an effect using the device, in one or more embodiments. The method comprises the following steps:
Step 922: Administer a psychedelic drug to a living body using a device (comprising an article of furniture).
Step 924: Monitor a physiological state of the living body using the device.
Step 926: Estimate an effect of the therapy.
Step 928: Modify the therapy based on a change in the physiological state.

In an embodiment, the massage therapy and the pressure therapy are provided using a blanket. In another embodiment, the blanket is at least one of a weighted blanket, a grounding mat, a magnetic therapy pad and a temperature adjustable blanket.

In an embodiment, the light therapy is provided using a light emitting device. The light therapy is designed to penetrate through the eyes and regulate the biological clock of the body through action on the hypothalamus. The hypothalamus controls the nervous system and the endocrine system, which together regulate all the biological functions of the human body. It sends information linked to light to the pineal gland, which informs other organs of it. The cells of the pineal gland, in turn, transform the nerve impulses, resulting in conversion of luminous messages into chemical molecules.

In an embodiment, the light emitting device can provide a non-chromatic light. In another embodiment, the light emitting device is operable to output a predetermined light frequency and duration.

In an embodiment, the visual based stress relief therapy is provided using a visual display.

In an embodiment, a combination of therapeutic stimuli is provided to the living body. In an embodiment, the device can provide a combination of therapies in a synchronized manner. For example, the device may combine color and sound therapy with aromatherapy to provide an enhanced synchronized mediated therapy designed to reduce stress and/or anxiety, and to treat symptoms of individuals suffering from the effects of stress and anxiety. The device can further synchronize specific frequencies of sound with that of color, light, and aroma to provide a more effective sensory experience.

FIG. 9D provides a flow chart of providing an aromatherapy in combination with a sound therapy using the device, in one or more embodiments. The method comprises the following steps:
Step 930: Provide a sound therapy to a living body using a device.
Step 932: Provide an aromatherapy to the living body using the device.
Step 934: Monitor a physiological state of the living body.
Step 936: Estimate an effect of the therapy.
Step 938: Modify the therapy based on a change in the physiological state.

FIG. 9E provides a flow chart of providing a psychedelic drug therapy in combination with an aromatherapy using the device, in one or more embodiments. The method comprises the following steps:
Step 940: Administer a psychedelic drug to a living body via a chair.
Step 942: Provide a sound therapy to the living body via the chair.
Step 944: Monitor a physiological state of the living body.
Step 946: Estimate an effect of the therapy.
Step 948: Modify the therapy based on a change in the physiological state.

FIG. 9F provides a flow chart of providing a psychedelic drug therapy in combination with a sound therapy using the device, in one or more embodiments. The method comprises the following steps:
Step 950: Provide an aromatherapy to a living body via a chair.
Step 952: Administer a psychedelic drug to the living body via the chair.
Step 954: Monitor a physiological state of the living body.
Step 956: Estimate an effect of the therapy.
Step 958: Modify the therapy based on a change in the physiological state.

FIG. 9G provides a flow chart for providing a psychedelic drug in combination with a sound therapy and an aromatherapy using the device, in one or more embodiments. The method comprises the following steps:
Step 960: Administer a psychedelic drug to a living body via a chair.
Step 962: Provide a sound therapy to the living body via the chair.
Step 964: Provide an aromatherapy to the living body via the chair.
Step 966: Monitor a physiological state of the living body.
Step 968: Estimate an effect of the therapy.
Step 970: Modify the therapy based on a change in the physiological state.

In an embodiment, the bio-monitoring system comprises a biofeedback control system and a vital monitoring system. In another embodiment, the therapy biofeedback control system modifies and controls a therapy session by changing a duration, an intensity and a type of therapeutic stimuli and combination thereof. In yet another embodiment, the biofeedback control system modifies and controls delivery of a drug to the living body.

The biomonitoring system is used to assess the physiological state of the living body to or as the treatment is being undertaken. The evaluation methods provide data which is electronically analyzed by a computing system having specialized software programs which are configured to establish treatment sessions and control or modify the therapy based on the physiological state.

In another embodiment, the method further comprises steps of storing data from a control unit of the device to a database; securing data access using a cyber security module; accessing the data from the database from a remote location via the cyber security module through authentication; and sending an instruction to the living body or a care provider via a communication module.

In an embodiment, the device may comprise a cyber security module, a communication module, a server, and a database.

In one aspect, a secure communication management (SCM) computer device for providing secure data connections in the healthcare environment is provided. The SCM computer device includes a processor in communication with memory. The processor is programmed to receive, from a first user computer device, a first data message from a user or an attendant. The first data message is in a standardized data format. The processor is also programmed to analyze the first data message for potential cyber security threats. If the determination is that the first data message does not contain a cyber security threat, the processor is further programmed to convert the first data message into a first data format associated with the healthcare environment and transmit the converted first data message to the healthcare system using a first communication protocol associated with the healthcare system.

According to an embodiment, secure authentication for data transmissions comprises, provisioning a hardware-based security engine (HSE) located in communications system, said HSE having been manufactured in a secure environment and certified in said secure environment as part of an approved network; performing asynchronous authentication, validation and encryption of data using said HSE, storing user permissions data and connection status data in an access control list used to define allowable data communications paths of said approved network, enabling communications of the communications system with other computing system subjects to said access control list, performing asynchronous validation and encryption of data using security engine including identifying a user device (UD) that incorporates credentials embodied in hardware using a hardware-based module provisioned with one or more security aspects for securing the system, wherein security aspects comprising said hardware-based module communicating with a user of said user device and said HSE.

In an embodiment, there is a cyber security module embedded in each of the layers namely Human Layer, Perimeter Layer, Network Layer, Endpoint Layer, Application Layer, Data Layer, and Mission Critical Layer. Each layer represents a different stage in network communication, from a human typing on a keyboard to the data system used for applications.

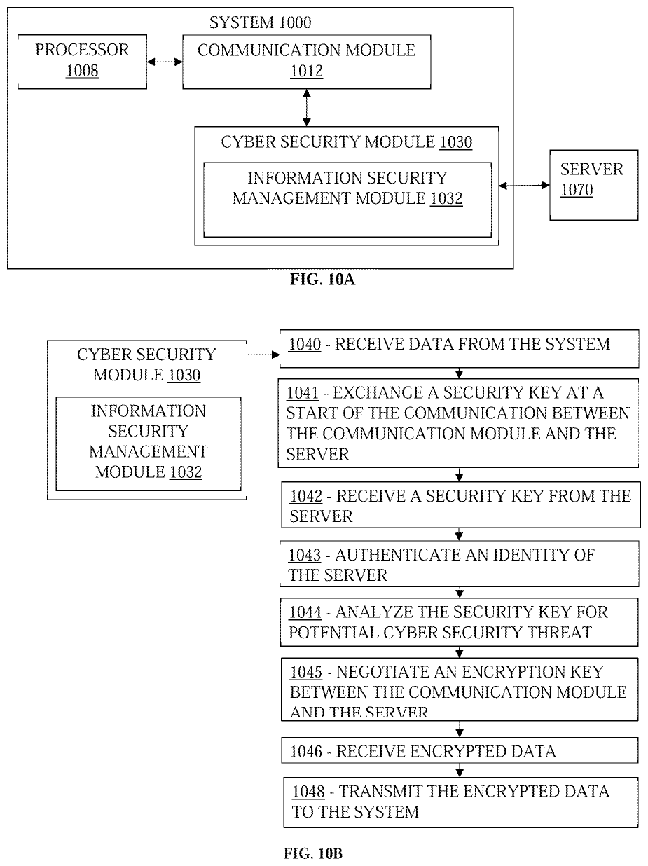

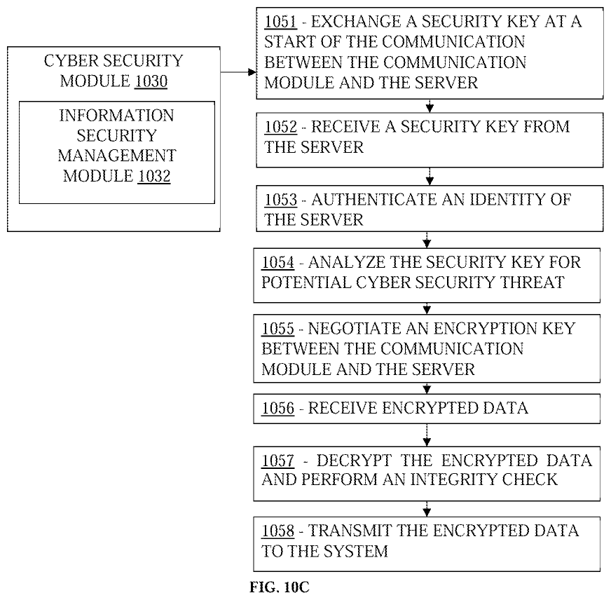

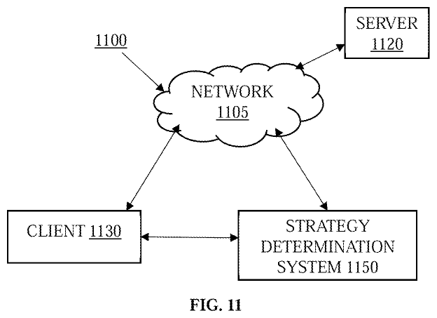

In an embodiment, FIG. 10A shows the block diagram of the cyber security module. The communication of data between the system 1000 and the server 1070 through the communication module 1012 is first verified by the information security management module 1032 before being transmitted from the system to the server or from the server to the system. The information security management module is operable to analyze the data for potential cyber security threats, encrypt the data when no cyber security threat is detected, and transmit the data encrypted to the system or the server.

In an embodiment, the cyber security module further comprises an information security management module providing isolation between the system and the server. FIG. 10B shows the flowchart of securing the data through the cyber security module 1030. At step 1040, the information security management module is operable to receive data from the system, for example, at least one of an input interface, the drug storage, and the database. At step 1041, the information security management module exchanges a security key at a start of the communication between the communication module and the server. At step 1042, the information security management module receives a security key from the server. At step 1043, the information security management module authenticates an identity of the server by verifying the security key. At step 1044, the information security management module analyzes the security key for potential cyber security threats. At step 1045, the information security management module negotiates an encryption key between the communication module and the server. At step 1046, the information security management module encrypts the data. At step 1047, the information security management module transmits the encrypted data to the server when no cyber security threat is detected.

In an embodiment, FIG. 10C shows the flowchart of securing the data through the cyber security module 1030. At step 1051, the information security management module is operable to: exchange a security key at a start of the communication between the communication module and the server. At step 1052, the information security management module receives a security key from the server. At step 1053, the information security management module authenticates an identity of the server by verifying the security key. At step 1054, the information security management module analyzes the security key for potential cyber security threats. At step 1055, the information security management module negotiates an encryption key between the system and the server. At step 1056, the information security management module receives encrypted data. At step 1057, the information security management module decrypts the encrypted data, performs an integrity check of the decrypted data. At step 1058, the information security management module transmits the decrypted data to the system, for example, at least one of output interface, drug storage, and the database through the communication module when no cyber security threat is detected.

In an embodiment, the integrity check is a hash-signature verification using a Secure Hash Algorithm 256 (SHA256) or a similar method. A cryptographic hash (sometimes called ‘digest’) is a kind of ‘signature’ for a text or a data file. SHA256 generates an almost-unique 256-bit (32-byte) signature for a text.

In an embodiment, the information security management module is configured to perform asynchronous authentication and validation of the communication between the communication module and the server.

In an embodiment, a perimeter network provides an extra layer of protection. In an embodiment, the perimeter network protects the system from a cyber security threat by using a plurality of firewalls. Usually, a perimeter network is the final step a packet takes traversing one of the system's networks on its way to the internet; and conversely the first network encountered by incoming traffic from the Internet to the system.

In an embodiment, a demilitarized zone (DMZ) network functions as a subnetwork containing an organization's exposed, outward-facing services. It acts as the exposed point to an untrusted network, commonly the Internet. A DMZ network will add an extra layer of security to an organization's local area network. It is a protected and monitored network node that faces outside the internal network and can access what is exposed in the DMZ, while the rest of the organization's network is safe behind a firewall. A DMZ Network gives organizations extra protection in detecting and mitigating security breaches before they reach the internal network, where valuable assets are stored. All services accessible to users on communicating from an external network can and should be placed in the DMZ, if one is used. The most common services include, but are not limited to, web servers, mail servers, file transfer protocol (FTP) servers.

In an embodiment, the information security management module is configured to raise an alarm if a cyber security threat is detected. In an embodiment, the information security management module is configured to discard the encrypted data received if the integrity check of the encrypted data fails.

In an embodiment, the information security management module is configured to check the integrity of the encrypted data by checking accuracy, consistency, and any possible data loss during the communication through the communication module.

In an embodiment, the information security management module is configured to perform asynchronous authentication and validation of the communication between the communication module and the server.

In an embodiment, the server is physically isolated from the system through the information security management module. When the system communicates with the server as shown in FIG. 10A, identity authentication is firstly carried out on the system and the server. The system is responsible for communicating/exchanging a public key of the system and a signature of the public key with the server. The public key of the system and the signature of the public key are sent to the information security management module. The information security management module decrypts the signature and verifies whether the decrypted public key is consistent with the received original public key or not. If the decrypted public key is verified, the identity authentication is passed. Similarly, the system and the server carry out identity authentication on the information security management module. After the identity authentication is passed on to the information security management module, the two communication parties, the system, and the server, negotiate an encryption key and an integrity check key for data communication of the two communication parties through the authenticated asymmetric key. A session ID number is transmitted in the identity authentication process, so that the key needs to be bound with the session ID number; when the system sends data to the outside, the information security gateway receives the data through the communication module, performs integrity authentication on the data, then encrypts the data through a negotiated secret key, and finally transmits the data to the server through the communication module. When the information security management module receives data through the communication module, the data is decrypted first, integrity verification is carried out on the data after decryption, and if verification is passed, the data is sent out through the communication module; otherwise, the data is discarded.

In an embodiment, the identity authentication is realized by adopting an asymmetric key with a signature.

In an embodiment, the signature is realized by a pair of asymmetric keys which are trusted by the information security management module and the system, wherein the private key is used for signing the identities of the two communication parties, and the public key is used for verifying that the identities of the two communication parties are signed.

In an embodiment, the identity authentication is that both communication parties need to authenticate their own identities through a pair of asymmetric keys, and a task in charge of communication with the information security management module of the system is identified by a unique pair of asymmetric keys.

In an embodiment, the dynamic negotiation key is encrypted by adopting an Rivest-Shamir-Adleman (RSA) encryption algorithm. RSA is a public-key cryptosystem that is widely used for secure data transmission. The negotiated keys include a data encryption key and a data integrity check key.

In an embodiment, the data encryption method is a Triple Data Encryption Algorithm (3DES) encryption algorithm. The integrity check algorithm is a Hash-based Message Authentication Code (HMAC-MD5-128) algorithm. When data is output, integrity check calculation is carried out on the data, the calculated Message Authentication Code (MAC) value is added with the head of the value data message, then the data (including the MAC of the head) is encrypted by using a 3DES algorithm, the head information of a security layer is added after the data is encrypted, and then the data is sent to the next layer for processing.

In an embodiment the next layer refers to a transport layer in the Transmission Control Protocol/Internet Protocol (TCP/IP) model.

In an embodiment, when the receiving side finds an authentication error or a MAC decryption error, it is necessary to send a fatal error message to the transmitting side and close the connection.

The information security management module ensures the safety, reliability, and confidentiality of the communication between the system and the server through the identity authentication when the communication between the two communication parties starts the data encryption and the data integrity authentication in the communication process. The method is particularly suitable for an embedded platform which has less resources and is not connected with a Public Key Infrastructure (PKI) system and can ensure that the safety of the data on the server of the drug storage cannot be compromised by hacker attack under the condition of the Internet by ensuring the safety and reliability of the communication between the system and the server in the system for smart storage.

In an embodiment, a system hardening strategy is implemented to prevent at least one attack. An attack graph analysis may be used to help analyze network vulnerability. Once an attack graph of conditions and/or exploits (e.g., at least one goal condition, at least one initial condition, at least one exploit) is obtained, allowable actions that may harden the conditions may be obtained. Costs associated with the allowable actions may also be obtained. Recommended actions to harden the network with respect to one or more goal conditions may be determined.

FIG. 11 is a system 1100 according to an embodiment of the invention. In this example, the system 1100 may comprise a network 1105 (e.g., the Internet, an intranet) wherein one or more computers 1120 (e.g., server, client) may communicate with one another. A strategy determination system 1150 may communicate with the client and/or the server. The strategy determination system 1150 may obtain an attack graph of conditions and/or exploits (e.g., using known techniques), obtain allowable actions that may remove one or more initial conditions to harden the network with respect to one or more goal conditions; obtain costs associated with the allowable actions, and determine recommended system hardening strategies to efficiently harden the network with respect to the goal condition(s), each system hardening strategy consisting of one or multiple allowable actions. As attackers may leverage complex interdependencies of network configurations and vulnerabilities to penetrate seemingly well-guarded networks, in an embodiment, the recommended actions may consider attacker exploits in isolation and/or in combination. Attack graphs may reveal such threats by enumerating potential paths that attackers can take to penetrate networks. This may help determine whether a given set of system hardening measures provides safety for given critical resources.

System hardening goal conditions may have a corresponding impact on removing paths in the attack graph. In addition, system hardening solutions that are optimal with respect to some notion of cost and/or time may be determined. Such system hardening solutions prevent the attack from succeeding, while minimizing the associated costs.

The strategy determination system 1150 may comprise: a determine allowable actions module; an associate costs module; a determine recommended actions module; or an approximation module; or any combination thereof. In the strategy determination method, an attack graph comprising conditions and/or exploits may be obtained, allowable actions that remove one or more initial conditions may be obtained, costs associated with the allowable actions may be obtained, and recommended strategies comprising allowable actions may be determined based upon costs and/or time constraints.

Spyware is a type of malware that may be installed on computers and collects bits of information at a time about users without their knowledge. The presence of spyware is typically hidden from the user and may be difficult to detect. Spyware programs may collect various types of personal information, such as Internet surfing habits and sites that have been visited but may also interfere with user control of the computer in other ways, such as installing additional software and redirecting Web browser activity.

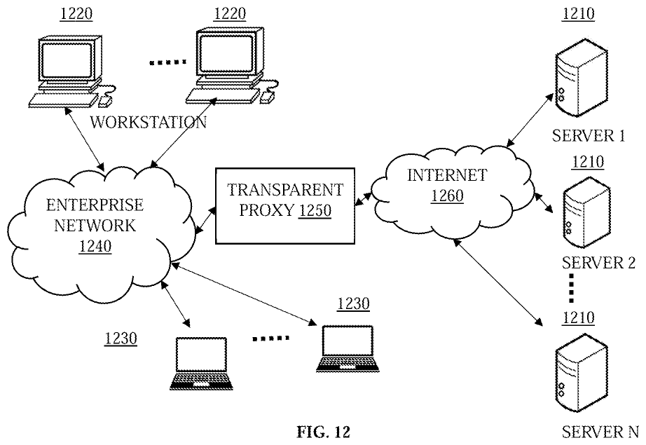

Passive detection may identify a fraction of the malware that is collected in an enterprise network but may not identify all of them. Embodiments of the present invention utilize active detection mechanism(s). The active detection mechanism(s) may also be called Active Content Challenges and may be implemented using a transparent proxy. FIG. 12 shows the architecture of a network using an embodiment of the transparent proxy 1250 in an Enterprise network 1240 including workstations 1220 and laptops 1230. The architecture may be fully transparent and may not require any application or network modifications both for client applications and servers and may accommodate various protocols including HTTP, encrypted HTTP (HTTPS) and Voice over IP (VOIP) protocols. The transparent proxy 1250 may mediate all traffic both encrypted and unencrypted when an application initiates a communication with a server 1210 connected to Internet 1260 outside the enterprise. Communication may pass through the firewall while being examined and analyzed by the transparent proxy 1250. According to an embodiment, a transparent proxy may be in a laptop or workstation. The transparent proxy may mediate all traffic both encrypted and unencrypted when an application initiates a communication with a remote server connected to the internet.

The transparent proxy 1250 may intercept outbound requests and issue Active Content Challenges to the requesting application. The principle is similar to Turing puzzles and Captchas, however, rather than trying to distinguish a human from software, the objective is to distinguish legitimate software from malware. Thus, unlike existing mechanisms that demand end-users to be involved in the identification process by solving a puzzle, the approach in this embodiment requires no user involvement or application modification. The transparent proxy for malware detection may include a monitor module, a protocol determination module, a challenge generation module, a response determination module, and a data control module. The transparent proxy may include interfaces for receiving and transmitting applications traffic and remote server traffic. The transparent proxy may be located on a network edge or on a laptop or workstation and may examine outgoing traffic. In general, the approach frustrates the communication of the malware by injecting traffic that the malware is incapable of parsing and generating a valid response contrary to the legitimate application.

In an embodiment, a secure virtual browsing environment is provided which includes creating a virtual browsing environment with a virtualized operating system sharing an operating system kernel of a supporting operating system and executing the browser application within the virtual browsing environment. Another embodiment includes receiving a website selection within a browser application, determining if the web site selection corresponds to a secure bookmark, and creating a second virtual browsing environment and executing the browser application within the second virtual browsing environment to access the website selection when the website selection corresponds to a website specified as a secure bookmark. Another embodiment includes monitoring operation of the operating system within the at least one virtual browsing environment, determining when the operation of the operating system includes potential malicious activity, and terminating the virtual browsing environment when the operation includes potential malicious activity.

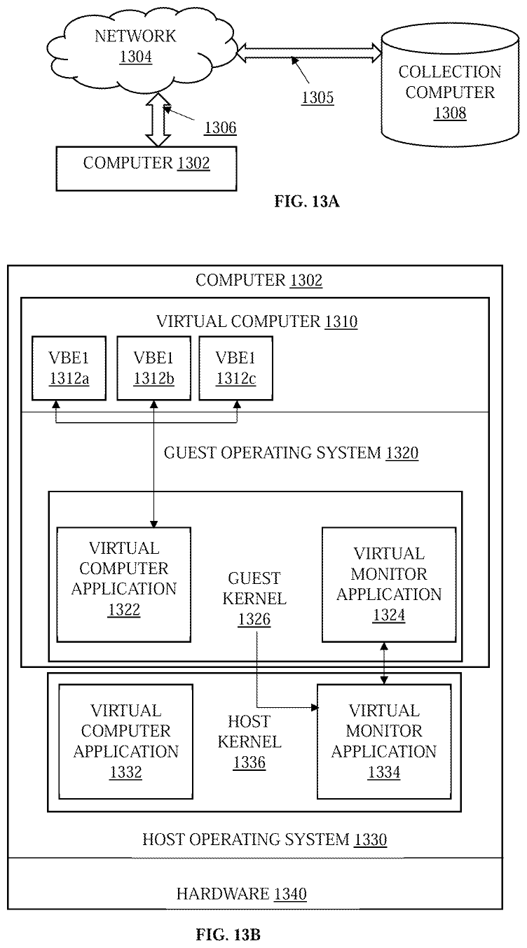

FIG. 13A illustrates a system 1300 for providing a virtual browsing environment according to one embodiment of the invention. As described below, embodiments of the system 1300 may provide a virtual browsing environment for executing a browser application on a computer. By executing the browser application within a separate virtual browsing environment, other applications, data, and modules of the computer may be protected from any malicious activity associated with the execution of the browser application. In addition, because in some embodiments only the browser application may be executed within the virtual browsing environment, malicious activity associated with the execution of the browser application may be easily detected. The system 1300 may include at least one computer 1302, at least one network 1304, and at least one collection computer (“CC”) 1308 and other components. The computer 1302 and the network 1304 may be connected by a connection 1306, and the network 1304 and the collection computer 1308 may be connected by a connection 1305. The collection computer 1308 may receive data from the network 1304 over the connection 1305. In some embodiments, the collection computer 1308 may also send data to the network 1304 or one or more computers or networks. The collection computer 1308 may also include hardware, such as one or more memory modules, one or more processors, and one or more input/output modules. In addition, the collection computer 1308 may include an operating system to manage the hardware. In some embodiments, the collection computer 1308 may also include a database that stores data received from the network 1304. The data included in the database may be stored in the collection computer's 1308 one or more memory modules, and the data may be managed by a database management application.

FIG. 13B illustrates the computer 1302 of FIG. 13A which includes a host operating system 1330 that provides an interface between the hardware 1340 and a user operating the computer 1302. The host operating system 1330 may be stored in the one or more memory modules and may be executed on the one or more processors included in the hardware 1340. The host operating system 1330 may include at least one host kernel 1336. The host kernel 1336 may manage the communication between the hardware 1340 and applications executed by the hardware 1340. The host kernel 1336 may use the virtual control application (VCA) 1334 to create and manage a virtual computer. Accordingly, the VCA 1334 may provide virtualization functionality. The host kernel 1336 may also include a shared preference directory 1332, which may store preferences for an application, such as a browser application. It should be understood that the one or more memory modules included in the hardware 1340 may store other applications besides those explicitly shown in FIG. 13B. In addition, the functionality provided by the applications stored in the one or more memory modules may be combined and distributed in various configurations.

In operation, as shown in FIG. 13B, the host kernel 1336 may execute the VCA 1334 to create a virtual computer 1310. The virtual computer 1310 may include its own guest host operating system 1320 with a guest kernel 1326. The guest operating system 1320 and guest kernel 1326 may operate similar to the host operating system 1330 and host kernel 1336. This type of virtualization where a generally complete copy of an operating system is provided within a virtual computer is generally referred to as “full virtualization.” Outside of the virtual computer 1310, the host operating system 1330 may continue to interact and manage the hardware 1340, while the guest operating system 1320 also may interact and manage the hardware 1340. Therefore, the virtual computer 1310 may create a second, isolated computing environment within the computer 1302. Each computing environment may execute different applications, access data from different locations in a memory module or from different memory modules, provide different operating systems, or combinations thereof. Creating the virtual computer 1310 may provide isolation between computing performed within the virtual computer 1310 and computing performed outside the virtual computer 1310 through the host operating system 1330. For example, the virtual computer 1310 may be unaware of any computing performed outside of the virtual computer 1310. Accordingly, an application executed within the virtual computer 1310 generally cannot access an application executed outside the virtual computer 1310.

As shown in FIG. 13B, the guest kernel 1326 may include a virtual computer control application (“VCCA”) 1322 and a virtual computer monitor application (“VCMA”) 1324. The VCCA 1322 may manage the operation of the virtual computer 1310. For example, as shown in FIG. 13B, the VCCA 1322 may create one or more virtual browsing environments (“VBE”) 1312 (e.g., VBE 1 1312 a, VBE 2 1312 b, and VBE 3 1312 c). Once created, the VCMA 1324 may monitor the operation of each VBE 1312 and may report each VBE's operation to the VCA 1334. To create a VBE 1312, the VCCA 1322 may use one or more virtualization modules or applications, such as OpenVZ, UnionFS patches, Solaris Zones, BSD Jail, or combinations thereof.

It is known that internet-enabled applications run side-by-side with all other desktop and system software with the privileges of the user. As a result, when a compromise occurs through the Internet, the entire system can be compromised by a single vulnerability in an Internet-enabled software such as a Web browser or an email client. By simply browsing to a Web page, a user can compromise their system, sometimes irreversibly.

In an embodiment, the system works by launching a virtual machine for each Internet-enabled or untrusted application that is started. The virtual machine provides a pristine guest operating system (OS) for the Internet-enabled or untrusted application that is launched. This operating system may be an operating system unmodified from the original version delivered by the manufacturer or another version suitably configured for the task of running intended applications. The virtual machine and its guest operating system may be temporally limited to exist only for the duration of the session of the application. When the user exits the application, the virtual machine can be destroyed. For the duration of the session, the virtual machine provides an isolated environment from the host machine from which it is launched. The virtual machine provides a level of isolation from the host machine that is the equivalent to running a physically separate machine from the host machine. Any attacks that occur on the machine via an Internet connection can compromise only the virtual machine that is started up for that session. When the session is terminated, so is the virtual machine and the compromise. With each new session, a pristine new virtual machine is started up, meaning that any malicious software that was downloaded or planted during a prior session is no longer present. The underlying host operating system does not need to maintain an Internet connection. As a result, Internet-based attacks have a very limited ability to compromise the host operating system.

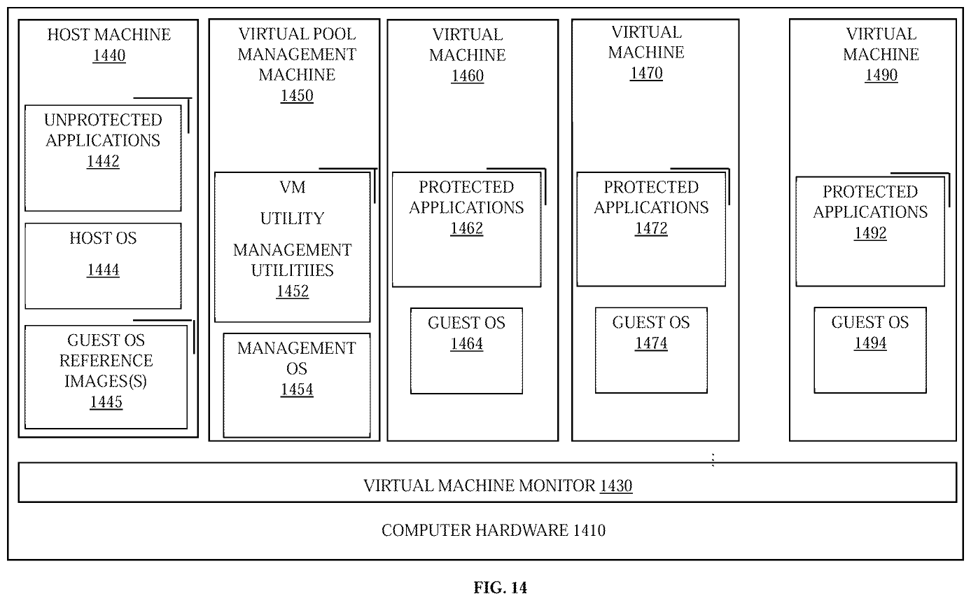

According to an embodiment, an architecture shown in FIG. 14 uses the standard virtual machine architecture with the Virtual Machine Monitor (VMM) 1430 running on the computer hardware 1410, and host operating systems (1444, 1454, 1464, 1474, and 1494) running on top of the VMM 1430. A host operating system (OS) 1444 is defined as the default machine the user normally uses and is the machine whose desktop is presented to the user. Guest OSs (1464, 1474 and 1494) are created by request when a protected application (1462, 1472 and 1492) is launched, or created in advance to enable higher performance when launching protected applications (1462, 1472 and 1492) into pre-instantiated guest OSs (1464, 1474 and 1494). A Management VM 1450 may be bootstrapped along with the Host OS 1444 and a reference guest OS image 1445 that is used for clones of the guest OS reference image 1445. The Management VM 1450 is used for command, control, and lifecycle maintenance of the guest OSs (1464, 1474 and 1494) based on the instructions from the host OS 1444. The number of guest OSs instantiated may be dependent on the number of protected applications launched and the performance limits of the underlying hardware. The VMM 1430 and VM 1450 should support live capture of the full system state in a file for subsequent replay. This file is called a “snapshot” of system state.

The host operating system 1444 may be configured for higher security so that it is unable to make Internet connections itself. The guest operating systems (1464, 1474 and 1494) may be free to make direct Internet connections; however, they should be restricted from freely accessing the host operating system 1444 by the virtual machine monitor 1430 that runs in its own hardware protection domain which provides hardware-equivalent strong isolation between the virtual machine and its host operating system. The guest operating systems (1464, 1474 and 1494), which are pristine builds of the OS, should also be “root secure”, which means that even if one of the guest operating systems (1464, 1474 and 1494) is compromised to a root user level or the kernel itself is compromised, the host operating system 1444 itself should not be compromised by the compromised guest operating system. Once a guest operating system is destroyed (upon closure of the protected application that started the guest OS), the compromise is now removed from the system.

As mentioned earlier, a reference guest OS image 1445 may be booted along with the host OS 1444. A snapshot of the reference guest OS image 1445 may be taken, then used to derive subsequent VM images by cloning it, i.e., creating a replica image of the reference guest OS. When a new untrusted application is to be started, a dispatch instruction is sent from the Host OS to the Virtual Pool Management Machine 1450, which then creates a VM for the application using the reference guest OS image, if the VM has not already been created. By cloning and pre-booting reference images, the response time for instantiating the application should be on par or even faster than the usual response time for starting a new application for users.

As described, FIG. 14 shows an embodiment of the present invention where virtual machines (VM) monitor 1430 runs directly on computer hardware 1410. In this embodiment, every host machine (1440, 1450, 1460, 1470 and 1490) is essentially a guest machine to the computer hardware. In this setup, the unprotected host applications 1442 run on the host machine 1440 natively and the host operating system 1444 runs these unprotected host applications 1442. In contrast, the guest virtual machines 1460, 1470 and 1490 run protected applications (1462, 1472, and 1492 respectively) that may talk to a network under guest operating systems (1464, 1474 and 1494 respectively).

The guest operating systems 1464, 1474, and 1494 are each cloned from one of the guest operating system images(s) 1445, and the images 1445 should be pristine snapshots of a running operating system. To increase speed, the snapshots may also include running applications. For example, an image 1445 of an operating system for an email virtual machine can include a copy of an email application running under the operating system.

The virtual pool management machine 1450 runs a series of virtual machine management utilities 1452 under a management operating system 1454. These utilities 1452 include functions that: create, destroy, put to sleep, and wake up virtual machines. The utilities also maintain a list that matches applications to virtual machines. In other embodiments, these same functions may be performed by pool management utilities running on a host machine.

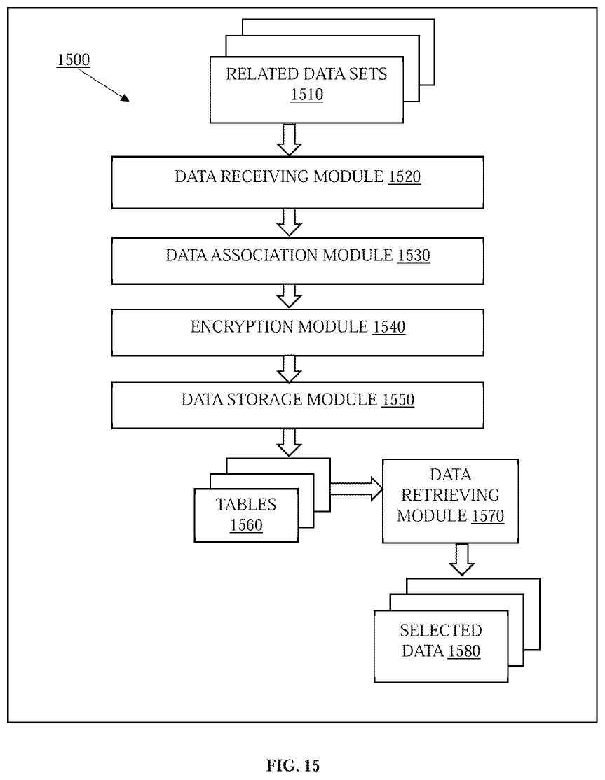

In an embodiment, sensitive data associations for related data values are protected. FIG. 15 is a block diagram of a system 1500 for protecting sensitive data associations according to an aspect of an embodiment of the present invention. The block diagram shows a multitude of modules. As shown, the system includes a data receiving module 1520 configured to receive a set(s) of related data values 1510. The set(s) of related data values 1510 preferably include at least a first data value and a second data value. The system normally operates against rule(s) that indicate which data value associations need to be kept secret. In the absence of such a rule, a default rule may be used such as the association of the first data value and the second data value needs to be kept secret.

A data association module 1530 may be configured to associate the first data value to a first data field; and the second data value to a second data field. An encryption module 1540 may then create first encrypted data by encrypting the first data value using a first encryption key; and create second encrypted data by encrypting the second data value using a second encryption key. A data storage module 1550 is configured to store: the first data value in a first data table 1560; the second data value in a second data table 1560; the first encrypted data in the second table 1560; and the second encrypted data in the first table 1560.

A data retrieving module(s) 1570 may be used to retrieve: the first data value by decrypting the first encrypted data using a first decryption key and/or the second data value by decrypting the second encrypted data using a second decryption key. As with the method embodiments, there are many possibilities for the encryption and decryption keys. The encryption key and the decryption key may be the same symmetric key. The encryption keys may be different or the same. Similarly, the decryption keys may be the same or different. The choice of keys should be made carefully to ensure that the data relationships in the rule(s) be kept secret. In some embodiments, the rule may be received from an external source. In the absence of an external rule, an internal rule or a default rule may be used.

In an embodiment, there is a tool for storing data records in a data store that is scalable and that allows a user to define their encryption and relieves a user from the task of managing keys used for data security. In an embodiment, application data and associated encryption key(s) are stored on at least k+1 remote servers using linear hashing (LH*) addressing. At least k+1 buckets are created on separate remote servers. At least k+1 key shares are generated for each of at least one encryption key. Each encryption key has a unique key number. Each key share is stored in a different key share record. Each of the key share records is stored in a different bucket using LH* addressing. Encrypted application data is generated by encrypting the application data with the encryption key(s). The encrypted application data is stored in encrypted data record(s). Each of the encrypted data records is stored in a different bucket among the buckets using LH* addressing.

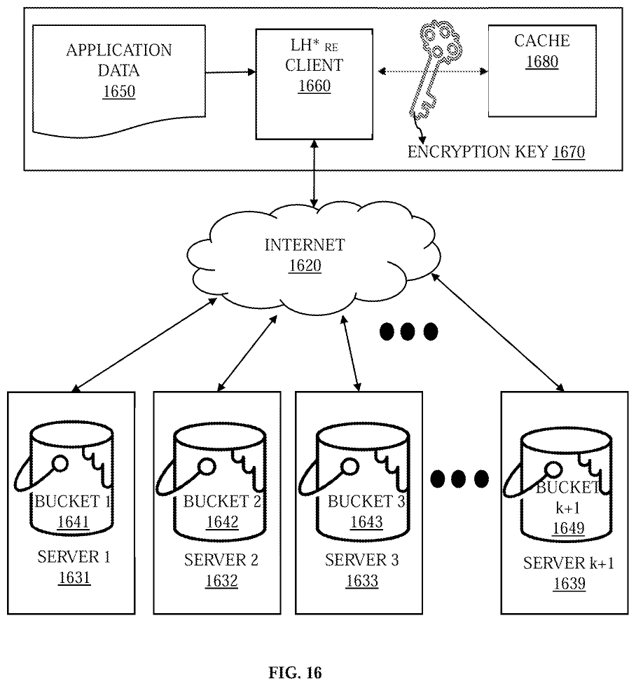

FIG. 16 is a system block diagram showing an example client 1610 interacting with k+1 remote servers (1631, 1632, 1633, . . . 1639) as per an aspect of an embodiment of the present invention. In these embodiments, one or more of clients (1610, 1611, . . . 1619) may have an LH*RE client 1610 configured to store a version of application data 1650 encrypted with an encryption key 1670 on remote servers (1631, 1632, 1633, . . . 1639). The remote servers (1631, 1632, 1633, . . . 1639) will likely be specialized servers configured to communicate with many client systems (1610, 1611 . . . 1619) and manage data buckets (1641, 1642, 1643, . . . 1649). The remote servers (1631, 1632, 1633, . . . 1639) may be geographically diverse. Some of the remote servers (1631, 1632, 1633, . . . 1639) may also be under the control of various organizations. In this way, the stored data may become harder for a third party to locate and retrieve all of the stored application data 1650 and key(s) 1670 from the data. Embodiments of the LH*RE client 1660 may be implemented as a computer readable storage medium containing a series of instructions that when executed by one or more processors on clients (1610, 1611, . . . 1619), causes the one or more processors to store application data 1650 on at least k+1 remote servers (1631, 1632, 1633, . . . 1639). In these embodiments, k is a freely set parameter of the system.

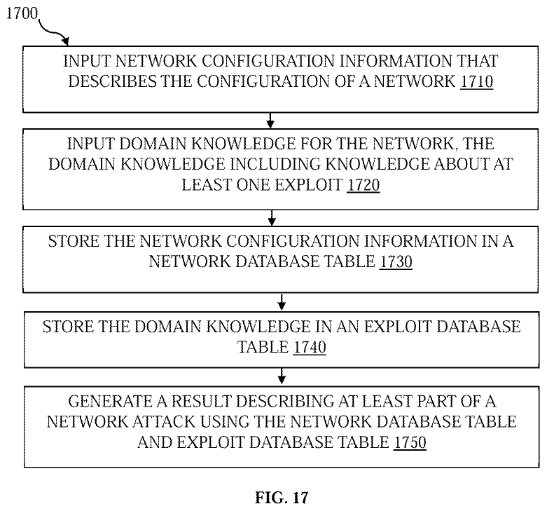

Attack graphs depict ways in which an adversary exploits system vulnerabilities in a network such as a computer network. Attack graphs may be important in defending against well-orchestrated network intrusions. FIG. 17 is a flow diagram of an aspect of an embodiment where the network configuration information input module is preferably configured to input network configuration information that describes the configuration of a network in 1710. The domain knowledge input module is preferably configured to input domain knowledge for the network in 1720. Domain knowledge may include knowledge about various exploits in the network. The network configuration information storage module is preferably configured to store network configuration information in at least one network database table in 1730. Similarly, the domain knowledge storage module is preferably configured to store the domain knowledge in at least one exploit database table 1740. The result generation module is preferably configured to generate a result using the network database table and exploit database table in 1750. The result may be generated in many ways.

In an embodiment, an Intrusion Detection System (IDS) is deployed on the system. An IDS is software and/or hardware designed to detect unwanted attempts at accessing, manipulating, and/or disabling computer systems, mainly through a network, such as the Internet. An intrusion detection system is used to detect malicious behaviors that can compromise the security of networked computer systems. An IDS may include Sensor(s) that are deployed at strategic locations in the network, which monitor traffic at the sensor location and generate security events upon detection of malicious behaviors; A central engine that records events (e.g., in a database) logged by the sensors; and Console(s) to monitor events and control the sensors. In some IDS implementations, all three components are combined in a single device or appliance. In a true distributed system, numerous sensors are deployed at various points in the network, which communicate over secure channels to the central engine. Multiple consoles may then interact with the central engine. In network-based intrusion detection systems (NIDS), sensors are located at monitoring points in a network. Traditionally, sensors may be placed at network borders or in a network demilitarized zone (DMZ), with the assumption that attacks are launched from outside the network to be defended. The sensor monitors network traffic at its point of deployment and analyzes the traffic content for patterns of malicious behavior.

Embodiments of the present invention locate the placement of intrusion detection system (IDS) sensors and prioritize IDS alerts using attack graph analysis. One embodiment predicts multiple ways of penetrating a network to reach critical assets. The set of such paths through the network constitutes an attack graph, which may be aggregated according to underlying network regularities, reducing the complexity of analysis. By knowing the paths of vulnerability through our networks, one may reduce the impact of attacks. IDS sensors may be placed to cover the attack graph, using a minimal number of sensors. This should minimize the cost of sensors, including effort of deploying, configuring, and maintaining them, while maintaining complete coverage of potential attack paths. An embodiment addresses the sensor placement as an instance of the non-deterministic polynomial-time (NP) hard minimal set cover problem using an efficient greedy algorithm. Once sensors are deployed and alerts are raised, a predictive attack graph may be used to prioritize alerts based on attack graph distance to critical assets.

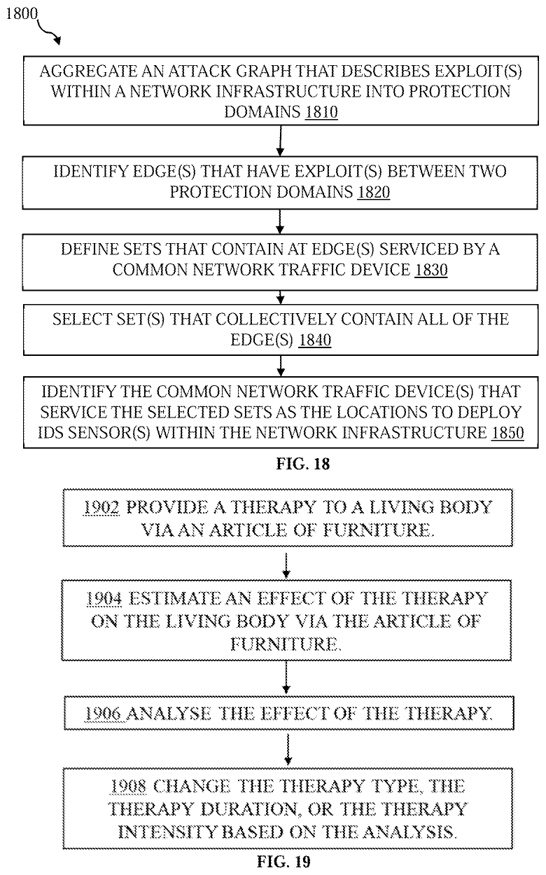

An embodiment of the present invention, as exemplified in FIG. 18, is a computer readable storage medium that contains instructions that when executed by at least one processor, causes the processor(s) to perform a method 1800 for identifying locations to deploy IDS sensor(s) within a network infrastructure. The method 1800 for identifying locations to deploy IDS sensor(s) within a network may comprise aggregating an attack graph that describes exploit(s) within a network infrastructure into protection domains 1810. The attack graph may be configured to describe exploit(s) in at least a part of the network infrastructure. Further, the embodiment may include identifying edge(s) that have exploit(s) between two protection domains 1820, defining sets that contain edge(s) serviced by a common network traffic device 1830, selecting set(s) that collectively contain all of the edge(s) 1840, and identifying the common network traffic device(s) that service the selected sets as the locations to deploy IDS sensor(s) within the network infrastructure 1850.

In an embodiment of the present invention, the selecting set(s) that collectively contain all of the edge(s) 1840 may further include selecting set(s) that cover critical path(s) through the network infrastructure that lead to a critical asset. The set selection method 1840 may further include selecting set(s) that cover critical path(s) through the network infrastructure that starts at an assumed threat source. Further variations of this embodiment may allow the set selection method 1840 to include selecting a minimal number of sensors necessary to cover critical path(s) through the network infrastructure. The set selection method 1840 may also further include utilizing a greedy algorithm. The greedy algorithm favors large sets that contain edge(s) that are infrequently used. Frequency is the number of times an edge appears across all sets.

In an embodiment of the present invention, the method 1800 for identifying locations to deploy on IDS sensor(s) within a network may further include prioritizing alerts from IDS sensors deployed within the network infrastructure using at least one attack graph distance to at least one critical asset. Attack graph distance may be measured in multiple ways such as: 1) the number of edges that are traversed to reach critical assets; 2) the number of protection domains crossed; and 3) the number of network traffic devices.

In an aspect, another method is described herein. The method comprises steps of providing a therapy to a living body via an article of furniture; and estimating an effect of the therapy on the living body via the article of furniture; wherein the article of furniture comprises a bio-monitoring system that monitors a physiological state of the living body.

In an embodiment, the therapy is provided using a therapeutic stimulus via the article of furniture. In another embodiment, the therapeutic stimulus is at least one of a psychedelic drug, a chemotherapy, a nutrient infusion, an audio stimulus, an aroma stimulus, a touch stimulus, a visual stimulus, and a light stimulus.

In an embodiment, the therapy type, the therapy duration, and the therapy intensity can be changed based on an analysis of the effect of the therapy on the living body.

FIG. 19 provides a flow chart of estimating an effect of the therapy on the living body via the article of furniture, in one or more embodiments. The method comprises the following steps:
Step 1902: Provide a therapy to a living body via an article of furniture.
Step 1904: Estimate an effect of the therapy on the living body via the article of furniture.
Step 1906: Analyze the effect of the therapy.
Step 1908: Change the therapy type, the therapy duration, or the therapy intensity based on the analysis.

### INCORPORATION BY REFERENCE

All references, including granted patents and patent application publications, referred herein are incorporated herein by reference in their entirety.
U.S. Pat. No. 10,837,194B2 titled “Portable relaxation pod”;
U.S. Pat. No. 11,013,883B2 titled “Stress reduction and sleep promotion system”;
U.S. Pat. No. 8,848,869B2 titled “Methods and devices for detecting, controlling, and predicting radiation delivery”;
US20100320819A1 titled “Chair and System for Transmitting Sound and Vibration”
U.S. Pat. No. 11,045,092 titled “Apparatus and method for measuring biologic parameters”;
US20180320394A1 titled “Modular Pod”;
US20160008568A1 titled “Relaxation apparatus and method”;
U.S. Pat. No. 5,387,178A titled “Multi-stimuli chair”;
U.S. Pat. No. 9,839,762B2 titled “System and method for reducing stress levels using color, sound, and aroma therapy”;
US20200410644A1 titled “Eye tracking method and apparatus”;
US20130261378 titled “Device for multisensory stimulation”;
US20180110939 titled “Method, system and apparatus for controlled delivery of opioid and other medications”;
U.S. Pat. No. 10,068,547B2 titled “Augmented reality surface painting”;
US20190050132A1 titled “Visual cue system”;
US20220015703A1 titled “Modular auricular sensing system”;
U.S. Pat. No. 9,203,861B2 titled “Methods and systems for determining hardening strategies”;
U.S. Pat. No. 9,436,822B2 titled “Virtual browsing environment”;
U.S. Pat. No. 10,956,184B2 titled “Malware detector”;
U.S. Pat. No. 9,846,588B2 titled “on demand disposable virtual work system”;
U.S. Pat. No. 8,082,452B2 titled “Protecting sensitive data associations”;
US20100054481A1 titled “Scalable distributed data structure with recoverable encryption”;
U.S. Pat. No. 8,566,269B2 titled “Interactive analysis of attack graphs using relational queries”;
US20100058456A1 titled “IDS sensor placement using attack graphs”.

1-121. (canceled)

122. A device comprising:
a drug delivery system;
a support surface to support at least a portion of a living body;
an enclosure to surround at least the portion of the living body;
a bio-monitoring system comprising a brain wave monitoring system and a facial expression recognizing system to monitor a physiological state comprising a mental state and an emotional state of the living body; and
a control system, wherein the control system is configured to adjust a psychological therapy based on a change in the physiological state;
wherein the psychological therapy comprises a combination therapy to the living body;
wherein the combination therapy comprises at least two of a psychedelic drug therapy, a chemotherapy, a nutrient infusion therapy, a sound therapy, an aromatherapy, a magnetic therapy, a massage therapy, a pressure therapy, a light therapy, and a visual based stress relief therapy.

123. The device of claim 122, wherein the device comprises an article of furniture.

124. The device of claim 122, wherein the support surface further comprises a blanket and wherein the blanket is at least one of a weighted blanket, a grounding mat, a magnetic therapy pad and a temperature adjustable blanket.

125. The device of claim 122, further comprises an armrest, wherein the armrest is foldable and is provided with a heating pad.

126. The device of claim 122, wherein the enclosure comprises an adjustable dome shaped housing that allows freedom of movement.

127. The device of claim 126, wherein the adjustable dome shaped housing further comprises a bridge that is foldable and provides a visual based stress relief therapy.

128. The device of claim 122, wherein the bio-monitoring system comprises a vital monitoring system.

129. The device of claim 122, wherein the bio-monitoring system comprises a biofeedback monitoring system and wherein an output from the biofeedback monitoring system controls a delivery of a drug by the drug delivery system.

130. The device of claim 122, further comprises an attachable device that comprises at least one of a sound emitting device, a light emitting device, an aroma infusion device, and an eye mask.

131. The device of claim 130, wherein the attachable device further comprises a computing system which can remotely receive and execute a control instruction.

132. The device of claim 122, wherein the device further comprises a control unit, wherein the control unit comprises an input module, a processor, a communication module, a database, a universal serial bus, a controller, a display, and a power module and wherein the control unit can remotely receive and execute a control instruction.

133. The device of claim 132, wherein the device can communicate data to a server via the communication module.

134. The device of claim 133, wherein the control unit further comprises a cyber security module.

135. The device of claim 122, wherein the device can be adjusted to an upright position, a reclined position, and a horizontal position.

136. The device of claim 122, wherein the device can navigate autonomously on a predefined track.

137. A method comprising steps of:
providing a psychological therapy to a living body using a device;
monitoring a physiological state comprising a mental state and an emotional state of the living body using the device; and
estimating an effect of the psychological therapy using a bio-monitoring system comprising a brain wave monitoring system and a facial expression recognizing system; and
modifying the psychological therapy based on a change in the physiological state; and
wherein the psychological therapy comprises a combination therapy to the living body;
wherein the combination therapy comprises at least two of a psychedelic drug therapy, a chemotherapy, a nutrient infusion therapy, a sound therapy, an aromatherapy, a magnetic therapy, a massage therapy, a pressure therapy, a light therapy, and a visual based stress relief therapy;
wherein the device comprises an article of furniture comprising a support surface to support at least a portion of the living body; an enclosure to surround at least the portion of the living body; and the bio-monitoring system that monitors the change in the physiological state of the living body.

138. (canceled)

139. The method of claim 137, the steps further comprise:
storing data from a control unit of the device to a database;
securing data access using a cyber security module;
accessing the data from the database from a remote location via the cyber security module through authentication; and
sending an instruction to the living body or a care provider via a communication module.

140. A method comprising:
providing a psychological therapy to a living body via an article of furniture; and
estimating an effect of the psychological therapy on the living body via the article of furniture; and
wherein the article of furniture comprises a bio-monitoring system comprising a brain wave monitoring system and a facial expression recognizing system that monitors a physiological state, the physiological state comprising a mental state and an emotional state of the living body;
wherein the psychological therapy comprises a combination therapy to the living body;
wherein the combination therapy comprises at least two of a psychedelic drug therapy, a chemotherapy, a nutrient infusion therapy, a sound therapy, an aromatherapy, a magnetic therapy, a massage therapy, a pressure therapy, a light therapy, and a visual based stress relief therapy;
and
adjusting the psychological therapy based on a change in the physiological state via the article of furniture.

141. The method of claim 140, wherein the bio-monitoring system further comprises a vital monitoring system.

142. A device comprising:
a drug delivery system;
a support surface to support at least a portion of a living body;
an enclosure to surround at least the portion of the living body; and
a bio-monitoring system comprising a brain wave monitoring system and a facial expression recognizing system to monitor a physiological state of the living body; and
a control system, wherein the control system is configured to adjust a therapy based on a change in the physiological state; and
wherein the therapy comprises a combination therapy to the living body.

143. The device of claim 142, wherein the combination therapy comprises at least two of a psychedelic drug therapy, a chemotherapy, a nutrient infusion therapy, a sound therapy, an aromatherapy, a magnetic therapy, a massage therapy, a pressure therapy, a light therapy, and a visual based stress relief therapy.
